(19) United States
(12) Patent Application Publication
(10) Pub. No.: US 2024/0264164 A1
(43) Pub. Date: Aug. 8, 2024

(54) DETERGENTS AND METHODS

(71) Applicants: OXFORD UNIVERSITY INNOVATION LIMITED, Oxford (GB); FREIE UNIVERSITAT BERLIN, Berlin (DE)

(72) Inventors: Leonhard Hagen URNER, Oxford (GB); Kevin PAGEL, Berlin (DE); Rainer HAAG, Berlin (DE); Dennis SHUTIN, Oxford (GB); Mark AGASID, Oxford (GB); Francesco FIORENTINO, Oxford (GB); Carol V. ROBINSON, Oxford (GB)

(21) Appl. No.: 18/560,091

(22) PCT Filed: May 11, 2022
(86) PCT No.: PCT/GB2022/051202

§ 371 Date: Nov. 9, 2023

(30) Foreign Application Priority Data
May 11, 2021 (GB) 2106700.4

(51) Int. Cl. G01N 33/68; C07C 43/13; C07H 15/04; C09K 23/42

(52) U.S. Cl. CPC G01N 33/6848; C07C 43/135; C07H 15/04; C09K 23/42

(57) ABSTRACT

A method of detecting a protein by mass spectrometry comprises: providing a solution comprising a hybrid detergent and a protein; providing a mass spectrometer comprising a nanoelectrospray ionisation source; vaporising the solution; ionising the protein; resolving the ionised protein; and detecting the resolved protein. The mass spectrometry methods may be used to interrogate the lipidome of a protein of interest, and/or for analysing membrane proteins in the form of complexes with ligands, and in particular lipids.

### FIELD OF THE INVENTION

The present invention relates to detergents and their use in methods for analysing proteins. More particularly, the present invention relates to hybrid detergents and their use in methods for preparing a protein sample, as well as methods for detecting proteins by mass spectrometry. The present invention also relates to methods for interrogating the lipidome of a protein of interest using mass spectrometry. The mass spectrometry methods are particularly suitable for analysing membrane proteins in the form of complexes with ligands, and in particular lipids.

### BACKGROUND TO THE INVENTION

Membrane proteins are embedded in biomembranes and their function is vital for every organism. Some of the most prevalent human diseases, including some cancers, result from their dysfunction. Despite representing around a third of the human genome, membrane proteins represent targets for more than half of all current therapeutic agents.

Membrane proteins are traditionally isolated with detergents, which consist of a hydrophilic (water-soluble) and hydrophobic (water-insoluble) part. Detergents interfere with protein-lipid and lipid-lipid interactions in membranes and shield hydrophobic protein surfaces from water by forming a proteomicelle. Unlike biomembranes, proteomicelles are water-soluble and can be enriched to high purity levels by chromatographic techniques. This facilitates the structural analysis of membrane proteins by biophysical techniques, such as X-ray crystallography, nuclear magnetic resonance (NMR) spectroscopy, or native mass spectrometry (native MS).

Biomembranes such as bacterial membranes can contain various lipid classes, e.g., phospholipids (PLs) and lipopolysaccharides (LPSs). Phospholipids have one or more phosphate head groups and include phosphatidylglycerol (PG), phosphatidylethanolamine (PE), and cardiolipin (CDL). In contrast, lipopolysaccharides (LPSs) have a phosphorylated lipid A core that is functionalised with up to six fatty acid chains and a polysaccharide head. The investigation of interactions between membrane proteins and lipids is important for the identification of antibiotic targets.

During proteomicelle formation, lipids are co-purified to varying degrees. In this way, detergents can also support the investigation of interactions between membrane proteins and native lipids. However, the co-purification of lipids with proteomicelles can also cause problems. First, lipids increase the heterogeneity of a sample which can cause difficulties when crystallising membrane proteins and with the resolution of spectral data obtained from membrane proteins. Second, a heterogeneous lipid composition may hamper a conclusive investigation of how individual lipids affect the structure and function of membrane proteins. Third, the removal of lipids from proteomicelles is stressing membrane proteins, which often precipitate when applying delipidation strategies rendering a structural analysis difficult.

The popularity of native MS in membrane protein research is rapidly advancing. In native MS, proteomicelles are transferred into the vacuum of a mass spectrometer most commonly by nanoelectrospray ionization (nESI). Detergent aggregates protect the native structure of membrane proteins during this process and are stripped off inside the mass spectrometer by thermal activation. Membrane protein ions are then further investigated by MS techniques to obtain information about mass, size, shape, subunit stoichiometry, and non-covalently bound ligands, such as drugs, nucleotides, and lipids. In particular, top down analysis using, e.g. Orbitrap mass spectrometers, allows the identification of lipids which dissociate from the protein inside of the mass spectrometer.

Native MS enables the investigation of protein-lipid interactions in the following ways:
(1) Membrane proteins are delipidated stepwise with detergents that exhibit weak delipidating properties, such as n-dodecyl-β-D-maltoside (DDM). Protein-lipid complexes are repetitively purified by immobilized metal ion affinity chromatography (IMAC), size-exclusion chromatography (SEC), or dialysis until MS data of sufficient quality are obtained. However, this process is time consuming, because the number of required purification repeats need to be determined by trial and error.
(2) Membrane proteins may be delipidated with strong delipidating detergents, such as n-octyl-ß-D-glucoside (OG) or tetraethylene glycol monooctyl ether (C8E4). Individual lipid classes are then added back to the sample to study their impact on the structure and function of membrane proteins. However, information about the natively interacting lipidome is lost once full delipidation is achieved and membrane proteins can precipitate during delipidation.

Often, the native lipidome that binds to a membrane protein is identified using workflow (1) and its impact on the structure and function of membrane proteins is studied using workflow (2).

Previously, progressive membrane protein delipidation for MS analysis has been achieved by either increasing the concentration of the detergent stepwise during purification or by a repeated detergent exchange in the same detergent. However, no design guidelines are available that allow the delipidating properties of all available detergents to be estimated. Consequently, suitable detergents are identified by trial and error and progressive delipidation protocols have to be individually optimized for every sample.

Detergents that are suitable for native MS are ideally not denaturing to membrane proteins in both solution and gas phase. However, it has previously been difficult to identify detergents with these properties. For instance, saccharide detergents can maintain folded states of membrane proteins in solution, but the energy required for the removal of saccharide detergents in the gas phase and Zₐᵥₑ values of released membrane protein ions are comparatively high which can cause unintended unfolding or dissociation of protein structures in the gas phase. In contrast, polyethylene glycol and amine oxide detergents often disturb the native fold of membrane proteins in solution, but the energy required for the gas-phase removal of the proteomicelle and the Zₐᵥₑ values of released membrane protein ions are comparatively low.

The ability to detect lipid-bound states varies with saccharide, polyoxyethylene glycol ether, and amino oxide detergents.

Dendritic oligoglycerol detergents (OGDs) have been shown to exhibit many advantageous properties when used in membrane protein mass spectrometry methods (see WO 2020/049294). However, there are some drawbacks to these detergents. In particular, the more mildly delipidating dendritic detergents are not suitable for detergent exchange using size exclusion chromatography and tend to give mass spectra with poorer signal-to-noise ratios.

According, there remains a need for methods which can be used for gradually delipidating and stabilising membrane proteins in a solution. There is also a need for detergents which may be used in such methods, and which can also be used for carrying out MS methods for detecting proteins.

### SUMMARY OF THE INVENTION

The present invention is based on the surprising discovery of a class of detergents which may stabilise membrane proteins in solution, enable the gradual delipidation of membrane proteins, and enable the native MS analysis of membrane protein-lipid complexes. During these processes, the native fold of the membrane protein may be maintained.

According to a first aspect of the invention there is provided a method of detecting a protein by mass spectrometry. The method comprises:
(a) providing a solution comprising a hybrid detergent and a protein;
(b) providing a mass spectrometer comprising a nanoelectrospray ionisation source, a mass analyser and a detector;
(c) vaporising the solution using the nanoelectrospray ionisation source;
(d) ionising the protein;
(e) resolving the ionised protein using the mass analyser; and
(f) detecting the resolved protein using the detector;
   wherein the hybrid detergent comprises a hybrid head group linked to a hydrophobic tail, wherein the hybrid head group comprises a first hydrophilic group and a second hydrophilic group which is different from the first hydrophilic head group, wherein each of the first and second hydrophilic groups is derived from a polyol or contains a charged group.

In a further aspect, a method of preparing a protein sample is provided. The method comprises:
(i) providing a solution which comprises an extraction detergent aggregate in which a protein is contained; and
(ii) contacting the extraction detergent aggregate with a hybrid detergent to give a solution which comprises a hybrid detergent aggregate in which the protein is contained, wherein the hybrid detergent has a structure as defined herein.

Also provided is a hybrid detergent as defined herein, as well as a solution comprising such a hybrid detergent and a protein.

A protein delipidation kit is also provided, said kit comprising at three different detergents, wherein at least one of the detergents is a hybrid detergent as defined herein.

In another aspect, the present invention provides a method of interrogating the lipidome of a protein of interest. The method comprises:
providing at least three solutions comprising a detergent and the protein, a different detergent being used in each solution;
providing a mass spectrometer comprising a nanoelectrospray ionisation source, a mass analyser and a detector, and for each of the solutions:
   vaporising the solution using the nanoelectrospray ionisation source;
   ionising the protein;
   resolving the ionised protein using the mass analyser;
   detecting the resolved protein using the detector; and
   determining the degree of lipidation in the detected protein;
calculating at least one of the hydrophobic-hydrophilic balance (HLB) and the packing parameter (p value) of each of the detergents; and correlating the HLB and/or p value of the detergents with the degree of lipidation in the detected protein.

### BRIEF DESCRIPTION OF THE DRAWINGS

FIG. 1a is a plot for the membrane protein AqpZ which shows the relative intensities of apo and protein-phospholipid complexes detected during nESI mass spectrometry against different detergents. FIG. 1b depicts the nESI mass spectra from which the relative intensities were determined.

FIG. 2a is a plot for the membrane protein AmtB which shows the relative intensities of apo and protein-phospholipid complexes detected during nESI mass spectrometry against different detergents. FIG. 2b depicts the nESI mass spectra from which the relative intensities were determined.

FIG. 3a is a plot for the membrane protein TSPO which shows the relative intensities of apo and protein-phospholipid complexes detected during nESI mass spectrometry against different detergents. FIG. 3b depicts the nESI mass spectra from which the relative intensities were determined.

FIG. 4 is a plot for the membrane protein MsCl which shows the relative intensities of apo and protein-lipopolysaccharide complexes detected during nESI mass spectrometry against different detergents.

FIG. 5a is a plot for the membrane protein AcrB which shows the relative intensities of apo and protein-lipopolysaccharide complexes detected during nESI mass spectrometry against the number of column volumes of detergent 1 used to delipidate the protein. FIG. 5b is a similar plot for AcrB which shows the relative intensities of apo and protein-lipopolysaccharide complexes detected during nESI mass spectrometry against the number of column volumes of hybrid detergent 3 used to delipidate the protein. FIG. 5c is a similar plot but for the membrane protein BtuCD which shows the relative intensities of apo and protein-lipopolysaccharide complexes detected during nESI mass spectrometry against the number of column volumes of detergent 1 used to delipidate the protein.

FIG. 6a shows the Coomassie and silver stain analysis of the membrane protein AcrB when delipidated using different column volumes of detergent 1. FIG. 6b shows a similar analysis but for the membrane protein BtuCD.

FIG. 7 is a plot for the membrane protein AmtB which shows that degree of phospholipid delipidation detected in nESI mass spectrometry is not dependent on the CAC of the detergent or Zₐᵥₑ charge state of the detected protein.

FIG. 8 depicts an idealised workflow for interrogating the lipidome of a membrane protein of interest.

FIG. 9 shows nESI mass spectra for the protein AqpZ obtained using a DDM detergent, a hybrid detergent, and a C8E4 detergent.

FIG. 10a depicts nESI mass spectra for the protein AqpZ obtained upon purification with DDM and delipidation with hybrid detergent 3 during different stages of ligand identification using top-down MS. FIG. 10b depicts similar spectra but for the protein AqpZ delipidated using hybrid detergent 5 (detergent signals labelled with an asterisk (*)).

FIG. 11a shows circular dichroism spectra obtained from solutions containing the membrane protein TSPO in different detergents. FIG. 11b shows circular dichroism spectra obtained from solutions containing the membrane protein AmtB in different detergents.

### DESCRIPTION OF VARIOUS EMBODIMENTS

### Definitions

For the purposes of the present invention, the following terms as used herein shall, unless otherwise indicated, be understood to have the following meanings.

The term “detergent” as used herein refers to a substance which lowers the surface tension of the medium in which it is dissolved, and/or the interfacial tension with one or more other phases. Detergents are generally amphipathic molecules, comprising both hydrophilic and hydrophobic groups, and may be anionic, cationic, non-ionic or zwitterionic unless otherwise specified.

The term “hydrophobic” as used herein refers to groups which associate with one another in an aqueous environment. Hydrophobic groups are non-polar by nature. In contrast, the term “hydrophilic” as used herein refers to groups which interact with water in an aqueous environment. Hydrophilic groups are polar by nature.

The term “hybrid head group” as used herein refers to a molecular structure which contains a first hydrophilic group and a second hydrophilic group which is different from the first hydrophilic head group. The first and second hydrophilic groups are not merely regioisomers of one another and, as such, they have different molecular weights. A “hybrid detergent” is a detergent which contains a hybrid head group.

The term “polyol” as used herein refers to a molecule containing at least two, and preferably at least 3 hydroxyl groups.

The term “charged group” as used herein refers to a group which may be anionic (negatively charged) or cationic (positively charged). A zwitterionic group contains both an anionic and a cationic charged group.

The term “oligomer” as used herein refers to a molecular structure that consists of identical repeating units, for instance 2 to 10 and preferably 2 to 5 repeating units.

The term “hydrocarbyl” as used herein refers to a group that consists only of carbon and hydrogen. Hydrocarbyl groups include straight chain and branched groups, cyclic and acyclic groups, and saturated and unsaturated groups. The term embraces groups which may contain a mixture of cyclic, acyclic, saturated and unsaturated groups. Unless otherwise specified, hydrocarbyl groups are unsubstituted and, as such, consist solely of carbon and hydrogen atoms. Preferred hydrocarbyl groups include alkyl, alkenyl, alkynyl and aryl groups which are described further below. The term “hydrocarbylene” as used herein refers to divalent groups.

The term “alkyl” as used herein refers to a saturated group which may be straight chain or branched, and cyclic or acyclic. The term embraces groups which are cycloalkyl groups, and groups which comprise cyclic and acyclic alkyl groups. Unless otherwise specified, alkyl groups are unsubstituted and, as such, consist solely of carbon and hydrogen atoms. The term “alkylene” refers to divalent groups.

The term “cycloalkyl” as used herein refers to a cyclic alkyl group. The term embraces monocyclic groups and polycyclic groups including fused rings structures and bridged ring systems. In some embodiments, cycloalkyl groups contain 3 to 20 carbon ring atoms. Cycloalkyl groups also include rings to which straight or branched chain acyclic alkyl groups as defined above are attached. Unless otherwise specified, cycloalkyl groups are unsubstituted and, as such, consist solely of carbon and hydrogen atoms. The term “cycloalkylene” refers to divalent groups.

The term “alkenyl” as used herein refers to an alkyl group, e.g. as described above, but which comprises at least one carbon-carbon double bond. Thus, the term embraces straight chain and branched alkenyl groups, as well as non-aromatic cycloalkenyl groups including polycyclic, such as fused ring and bridged ring, structures. Alkenyl groups are preferably, but not necessarily, bonded to the rest of a molecule through a carbon which forms part of a double bond. Unless otherwise specified, alkenyl groups are unsubstituted and, as such, consist solely of carbon and hydrogen atoms. The term “alkenylene” refers to divalent groups.

The term “alkynyl” as used herein refers to an alkyl group, e.g. as described above, but which comprises at least one carbon-carbon triple bond. Thus, the term embraces straight chain and branched alkynyl groups. Alkynyl groups are preferably, but not necessarily, bonded to the rest of a molecule through a carbon which forms part of a triple bond. Unless otherwise specified, alkynyl groups are unsubstituted and, as such, consist solely of carbon and hydrogen atoms. The term “alkynylene” refers to divalent groups.

The term “aryl” as used herein refers to an aromatic ring system in which each of the ring members is carbon. In some embodiments, aryl groups include 6 to 20 ring members. Aryl groups include, but are not limited to, groups such as phenyl and naphthyl. Unless otherwise specified, aryl groups are unsubstituted. The term “arylene” refers to divalent groups.

The term “heterocyclyl” as used herein refers to an aromatic or non-aromatic ring system in which one or more ring members is a heteroatom such as, but not limited to, N, O, and S. A heterocyclyl ring may include one or more double bonds, and so a heterocyclyl group can be a cycloheteroalkyl or a heteroaryl group or, if polycyclic, any combination thereof. In some embodiments, heterocyclyl groups include 3 to 20 ring members. The term “heterocyclyl group” includes polycyclic ring systems containing a heteroatom in the ring and includes fused ring species including those comprising fused aromatic and non-aromatic groups. Unless otherwise specified, heterocyclyl groups are unsubstituted. The term “heterocyclylene” refers to divalent groups.

The term “cycloheteroalkyl” as used herein refers to a cycloalkyl group, e.g. as described above, but in which one or more ring members is a heteroatom such as, but not limited to, N, O, and S. Thus, the term embraces polycyclic, such as fused ring and bridged ring, structures. In some embodiments, cycloheteroalkyl groups include 3 to 20 ring members. Unless otherwise specified, cycloheteroalkyl groups are unsubstituted. The term “cycloheteroalkylene” refers to divalent groups.

The term “heteroaryl” as used herein refers to an aromatic ring system in which one or more ring members is a heteroatom such as, but not limited to, N, O, and S. In some embodiments, heteroaryl groups include 5 to 20 ring members. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Unless otherwise specified, heteroaryl groups are unsubstituted.

The term “heteroarylene” refers to divalent groups.

The term “substituted” as used herein in connection with a chemical group means that one or more (e.g. 1, 2, 3, 4 or 5) of the hydrogen atoms in that group are replaced independently of each other by a corresponding number of substituents. It will, of course, be understood that the one or more substituents may only be at positions where they are chemically possible, i.e. that any substitution is in accordance with permitted valence of the substituted atom and the substituent and that the substitution results in a stable compound. The term is contemplated to include all permissible substituents of a chemical group or compound.

### Hybrid Detergents

The hybrid detergents used in the present invention comprise a hybrid head group linked to a hydrophobic tail. The hybrid detergent preferably has the formula:

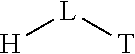

where: H represents the hybrid head group;
L represents a linking group; and
T represents the hydrophobic tail.

The hybrid head group comprises a first hydrophilic group and a second hydrophilic group which is different from the first hydrophilic head group.

Each of the first and second hydrophilic groups is derived from a polyol or contains a charged group. Preferably, at least one, for example both, of the first and second hydrophilic groups is derived from a polyol.

Suitable polyols may be selected from diols, triols and saccharides; oligomers of diols, triols and saccharides; or combinations thereof. For instance, the polyol from which the first and second hydrophilic groups may be derived may contain an oligomer of a diol, triol or saccharide linked to a different oligomer of a diol, triol or saccharide.

In some instances, the polyol is a diol, a triol or a saccharide; an oligomer of a diol, triol, saccharide, or combinations.

The diol may be a C₁₋₁₀ diol, preferably a C₂₋₈ diol, and more preferably a C₃₋₅ diol, such as ethylene glycol or propylene glycol. Particularly preferred as a diol is ethylene glycol.

The triol may be a C₂₋₁₀ triol, preferably a C₃₋₈ triol, and more preferably a C₃₋₆ triol. Particularly preferred as a triol is glycerol. Where an oligomer of glycerol is present, this is preferably not a dendritic oligomer (e.g. as described in WO 2020/049294) but is rather a 1,3 oligoglycerol.

The saccharide will generally be a monosaccharide, for instance selected from tetroses (i.e. saccharides containing 4 carbon atoms), pentoses (i.e. saccharides containing 5 carbon atoms) and hexoses (i.e. saccharides containing 6 carbon atoms). Preferably, the saccharide is selected from pentoses and hexoses, and more preferably from hexoses such as glucose and fructose.

The saccharide may be in a cyclic or linear form, though preferably it is in a cyclic form. Where the saccharide is an oligosaccharide, the saccharide monomers will generally be joined by 1-4 glycosidic links.

In specific instances, the polyol may be selected from:

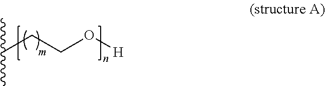

where: m is 1-3, preferably 1-2, and more preferably 1, and
n is 1-8, preferably 1-6, and more preferably 1-4;

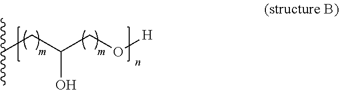

where: each m is independently 1-3, preferably 1-2, and more preferably is 1, and
n is 1-5; preferably 1-3, and more preferably 1-2;

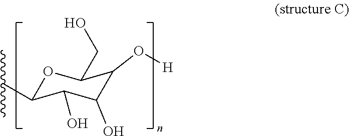

where: n is 1-5, preferably 1-3, and more preferably 1-2; and

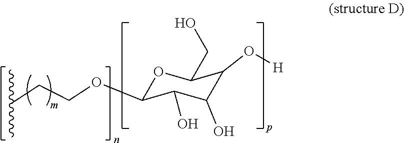

where: m is 1-3, preferably 1-2, and more preferably 1,
n is 1-5, preferably 1-3, and more preferably 1-2; and
p is 1-5, preferably 1-3, and more preferably 1-2.

The first and second hydrophilic groups are preferably derived from different polyol structural classes. Thus, the first and second hydrophilic groups are preferably not both derived from structure A. The first and second hydrophilic groups are preferably not both derived from structure B. The first and second hydrophilic groups are preferably not both derived from structure C. The first and second hydrophilic groups are preferably not both derived from structure D.

The first and second hydrophobic groups are derived from polyols and, as such, the polyol groups—once present in the hybrid head group—may contain one or more substituents in place of an —OH group. Thus, in some embodiments, the one or more—OH groups in the polyol are optionally substituted with a group selected from: —NR₂, —N+R₃, —N⁺R₂O⁻, —C(O)NR₂, —OC(O)NR₂, —NRC(O)NR₂, —NRC(S)NR₂, —C(NR)NR₂, —C(R)═NR, —CR═N—NR₂, —C═N—NRC(O)R, —C(O)R, —C(O)OR, —C(O)O⁻, —OC(O)R, —OC(O)OR, —OC(O)O⁻, —OS(O)₃⁻, —OS(O)₂OR, —OS(O)R, —S(O)₃⁻, —S(O)₂OR, —S(O)R, —S—SR, —OP(O)(OR)₂, —OP(O)(OR)O⁻, —PR₃, —P(O)(OR)₂, —P(O)(OR)O⁻.

Each R is independently selected from H and C₁₋₄ alkyl, preferably from H and C₁₋₂ alkyl, and more preferably from H and methyl.

However, it is generally preferred that the first and/or second hydrophilic groups are selected from (rather than derived from) polyols, such as the polyol groups described above.

Where the first and/or second hydrophilic groups contains a charged group, the charged group may be selected from nitrogen-containing groups, sulfur-containing groups, oxygen-containing groups, phosphate-containing groups, and combinations thereof. Preferably, the charged group is a phosphate-containing group.

Where the first and/or second hydrophilic groups contains a charged group, the first and/or second hydrophilic groups preferably contain from 1-10 carbon atoms, preferably from 2-8 carbon atoms, and more preferably from 3-5 carbon atoms.

Where the first and/or second hydrophilic groups contains a charged group, the first and/or second hydrophilic groups may be derived from a lipid head group. For instance, the first and/or second hydrophilic groups may be selected from:

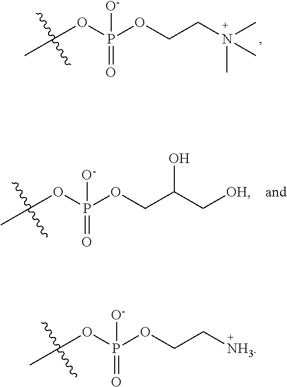

In preferred embodiments, the hybrid head group has the structure:

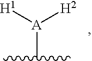

where: A represents a trivalent group;
H¹ represents the first hydrophilic group; and
H² represents the second hydrophilic group,

The trivalent group, A, is not particularly limited. Preferably the trivalent group contains from 1 to 15 carbon atoms, preferably from 2 to 10, and more preferably from 3 to 6 carbon atoms.

The trivalent group may be derived from a polyol (e.g. glycerol, a saccharide, or a benzenetriol), optionally linked to one or more spacing groups, for instance once spacing group. Suitable spacing groups include heteroarylene and arylene groups in which one of the hydrogen groups on the ring is substituted with oxygen (—O—) such that the oxygen provides one of the valent groups in trivalent group A.

Preferably the trivalent group is derived from a polyol, and more preferably from glycerol. Glycerol-derived trivalent groups are particularly compatible with mass spectrometry methods. Thus, in preferred embodiments, the hybrid head group has the structure:

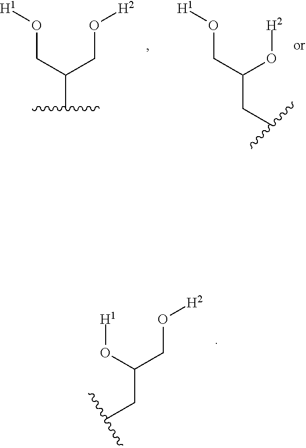

Particularly preferred is a hybrid head group having the structure:

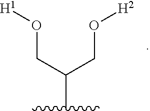

The hybrid head group preferably contains at least 2 hydroxy groups. Without wishing to be bound by theory, it is believed that hydroxy groups stabilise the protein-detergent complex. However, if the stability of the protein-detergent complex is too high, then it can be difficult to release the protein during mass spectrometry. The hybrid head group preferably contains up to 7, more preferably up to 6, such as up to 5 hydroxy head groups. Thus, the hybrid head group may contain from 2 to 7, preferably from 2 to 6, and more preferably from 2 to 5 hydroxy groups.

The linking group, L, that connects the hybrid head group to the hydrophobic tail may be selected from: hydrocarbylene, heterocyclylene, O, S, NR′, NR′—O, C(O)NR′, OC(O)NR′, OC(O)O, NR′C(O)NR′, NR′C(S)NR′, C(NR′)NR′, C(O), C(O)O, S(O)₂, S(O), S(O)₂O, S—S, CR′═N, CR′═N—NR′, C═N—NR′C(O), and combinations of up to three of these groups, where each R′ is independently selected from H, C₁₋₄ alkyl and C₁₋₄ alkoxy.

It will be appreciated that these groups may be used to link the hybrid head group and the hydrophobic tail of the hybrid detergent in any orientation. For instance, the group NR′—O may be used as: H—NR′—O-T or as H—O—NR′—T.

Suitable hydrocarbylene groups include alkylene, alkenylene, alkynylene and arylene groups, preferably C₁₋₆ alkylene, C₂₋₆ alkenylene, C₂₋₆ alkynylene and C₅₋₁₀ arylene, and more preferably C₁₋₃ alkylene. C₂₋₃ alkenylene, C₂₋₃ alkynylene and C₅₋₆ arylene. The alkylene, alkenylene and alkynylene groups are preferably acyclic.

Suitable heterocyclylene groups include 5-10 membered, and preferably 5-6 membered, heterocyclylene rings containing 1 or 3 heteroatoms. The heteroatoms in the heterocyclylene rings are preferably selected from O, N and S, and more preferably from O and N. The heterocyclene groups may be selected from heteroalkylene and heteroarylene groups, and preferably from heteroarylene groups. Preferred groups include those derived from triazole, imidazole, oxazole and pyridine, i.e. divalent forms of these groups.

R′ is preferably selected from H, C₁₋₂ alkyl and C₁₋₂ alkoxy, and more preferably from H and C₁₋₂ alkyl.

The linking group is preferably selected from O, S, C(O)O, O—C₁₋₄ alkylene-aryl and OC(═O)NR′, and is preferably O.

The hybrid detergents comprise a hydrophobic tail. It is this group which is believed to associate with hydrophobic portions on the surface of proteins.

The hydrophobic tail may be a C₆₋₁₀₀ alkyl group in which one or more methylene groups may be independently replaced by a unit selected from: C₂₋₆ alkenylene, C₂₋₆ alkynylene, C₅₋₁₀ arylene, O, S, NR″, NR″—O, C(O)NR″, OC(O)NR″, OC(O)O, NR″C(O)NR″, NR″C(S)NR″, C(NR″)NR″, C(O), S(O)₂, S(O), S(O)₂O, S—S, CR″═N, CR″═N—NR″, C═N—NR″C(O), where each R″ is independently selected from H, C₁₋₄ alkyl and C₁₋₄ alkoxy.

Preferably, the hydrophobic tail is a C₈₋₅₀ alkyl group, such as a C₁₀₋₃₀ alkyl group, in which one or more methylene groups may be independently replaced by a unit as described above.

The hydrophobic tail may have up to 6 methylene groups, preferably up to 4 methylene groups, and more preferably up to 2 methylene groups replaced by a unit as described above.

The hydrophobic tail preferably comprises a terminal acyclic alkyl group having at least 6 carbon atoms. This group may be branched or unbranched but, where it is branched, it preferably only comprises methyl side chains, e.g. 1 to 4, e.g. 1 or 2, methyl side chains. R″ is preferably selected from H, C₁₋₂ alkyl and C₁₋₂ alkoxy, and more preferably from H and C₁₋₂ alkyl.

In some embodiments, the hydrophobic tail may be lipid-like. For instance, the hydrophobic tail may be derived from a lipid. Hydrophobic tails derived from a lipid include those derived from sterols, such as cholesterol. Lipid-like hydrophobic tails may also be structurally similar to lipids, such as fatty acids. For example, a lipid-like hydrophobic tail may comprise at least one group having the structure—OC(O)—C₁₀₋₃₀ acyclic alkyl. A cholesterol hydrophobic tail has the structure:

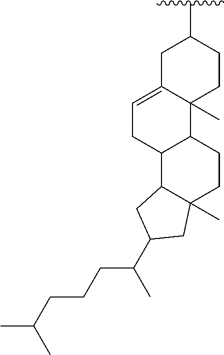 [SMILES: *C1CCC2(C)C(=CCC3C4CC(C(C)CCCC(C)C)CC4(C)CCC32)C1]

The hydrophobic tail is preferably selected from C₁₀₋₃₀ acyclic alkyl, and more preferably from C₁₀₋₂₀ acyclic alkyl.

The hybrid detergent preferably has a total number of carbons of up to 60, for instance up to 50 carbon atoms, such as up to 40 carbon atoms.

The hybrid detergent preferably has a molecular weight of up to 1,000 Da, preferably up to 800 Da, and more preferably up to 650 Da.

The hybrid detergents of the present invention are particularly useful in gradually delipidating membrane proteins. Without wishing to be bound by theory, it is believed that the degree to which phospholipids are removed from membrane proteins is dependent on the hydrophobic-lipophilic balance (HLB) and the packing parameter, p, of the detergents. While a higher HLB leads to less delipidation, a higher p generally leads to more delipidation.

The hybrid detergent preferably has an HLB of at least 11. The hybrid detergent preferably has an HLB of up to 15. Thus, the hybrid detergent may have an HLB of from 11 to 15. The HLB is a known property of detergents that is calculated according to the following equation:

$$HLB = 20 \times ( 1 - {MW}_{tail} / MW )$$

where: MW represents the molecular weight of the detergent; and
MWₜₐᵢₗ represents the molecular weight of the hydrophobic tail.

The hybrid detergent preferably has a p value of at least 0.08. The upper limit of the p value is not particularly restricted, though the p value will typically be up to 0.33. Preferred hybrid detergents of the present invention generally have a p value of up to 0.15. Thus, the hybrid detergent may have a p value of 0.08 to 0.33 such as from 0.08 to 0.15. The p value is a known property of detergents that is calculated according to the following equation:

$$p = {V}_{tail} / ( {I}_{tail} \times {A}_{head} )$$

where: Vₜₐᵢₗ represents the volume of the hydrophobic tail;
Iₜₐᵢₗ represents the length of the hydrophobic tail; and
A_{head} represents the area of the head group that is occupied at the interface between the detergent aggregate and solvent.

The values for Vₜₐᵢₗ, Iₜₐᵢₗ and A_{head} may be calculated using methods known in the art, such as those mentioned in the examples.

### Detergent Libraries (Kits)

As discussed in more detail below, the hybrid detergents of the present invention may be used as part of a detergent library which is useful for the gradual delipidation of membrane proteins. Thus, the present invention provides a protein delipidation kit, said kit comprising at least three different detergents. At least one of the detergents is a hybrid detergent as described herein.

In some embodiments, the kit contains a library of detergents that has been prepared using a combinatorial synthesis method described herein. Thus, the kit may comprise three detergents which are identical apart from the hydrophilic groups in their head group: the first detergent is a hybrid detergent as described herein having a first hydrophilic group and a second hydrophilic group which is different from the first hydrophilic group in its head group (i.e. an H¹H² head group); the second detergent has two first hydrophilic groups in its head group (i.e. an H¹H¹ head group); and the third detergent has two second hydrophilic groups in its head group (i.e. an H²H² head group).

The kit may comprise a further two detergents which are identical to one another apart from the hydrophilic groups in their head group: the fourth detergent is a hybrid detergent as described herein having a first hydrophilic group and a second hydrophilic group which is different from the first hydrophilic group in its head group, wherein at least the first hydrophilic group is different from the first and second hydrophilic groups of the first detergent (i.e. an H¹H² head group); and the fifth detergent has two first hydrophilic groups in its head group (i.e. an H¹′H¹′ head group). If H²′ is different from the first and second hydrophilic groups of the first detergent, then the kit may comprise a sixth detergent which is identical to the fourth and fifth detergents except for it has two second hydrophilic groups in its head group (i.e. an H²′H²′ head group).

The kit may comprise instructions for carrying out a method in which the detergents are used, such as a mass spectrometry method or a protein preparation method as defined herein.

### Preparation of the Hybrid Detergents

The hybrid detergents of the present invention may be prepared using standard techniques in the art, e.g. using standard addition reactions between a hybrid head group, a linking group and a hydrophobic tail.

However, preferred hybrid detergents may be synthesised by a method which comprises a head group synthesis stage, followed by a stage in which a hydrophobic tail is added. For instance, the hybrid detergents may be synthesised as follows:

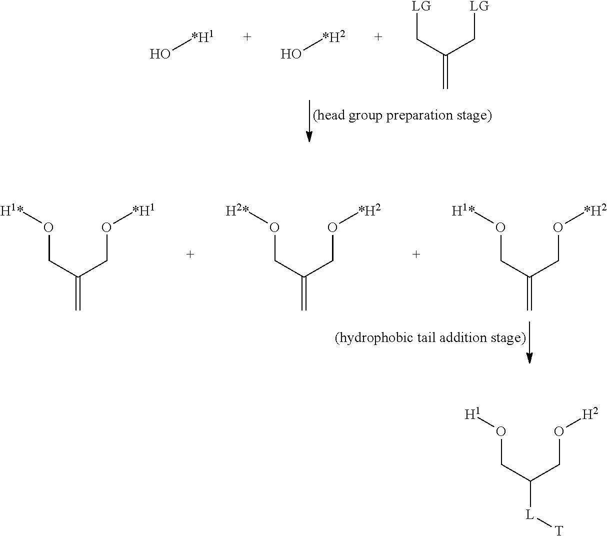

where: LG represents a leaving group; and
represents that the head groups H¹ and H² are in a protected form.

Leaving groups are well-known in the art. Suitable leaving groups may be selected from halides (e.g. C₁, Br and I), substituted aryloxy groups (e.g. —O—Ar, where Ar is selected from nitro-substituted aryl groups such as p-nitrophenyl), and sulfonates (e.g. —OSO₂A, where A is selected from tolyl, methyl, —CF₃, —CH₂Cl, phenyl and p-nitrophenyl). Preferred leaving groups are selected from Cl and Br.

Protecting groups are also well-known in the art. For instance, alcohols may be protected by benzyl protecting groups or, where two hydroxy groups are on neighbouring carbon atoms, by a ketone (e.g. propan-2-one) so as to form an acetal.

In a first preparation method, the protected head groups are separated from one another, e.g. by chromatography, after the head group preparation stage and the protected hybrid head group is converted into a hybrid detergent in the hydrophobic tail addition stage. The conditions under which the first preparation method are carried out may be controlled to promote formation of the hybrid head group over other head groups and, as such, the first preparation method may be used to prepare a hybrid detergent in relatively high yields.

In a second preparation method, the protected head groups are not separated from one another after the head group preparation stage and are together converted into detergents in the hydrophobic tail addition stage. The detergents and then preferably separated from one another, e.g. by chromatography. This combinatorial synthesis is particularly useful when a detergent library is desired.

### Mass Spectrometry Methods

The hybrid detergents may be used in a method of detecting a protein by mass spectrometry. The method involves the use of a solution comprising a hybrid detergent as defined herein and a protein. The solution is vaporised using a nanoelectrospray ionisation source. The protein is ionised, and subsequently resolved and detected.

Proteins may be composed of one (mono) or more (multi) associated polypeptide chains. Thus, the protein may be a monomeric or a multimeric protein, for example an oligomeric membrane protein. Oligomeric proteins include both homooligomeric (identical polypeptide chains) and heterooligomeric (different polypeptide chains) proteins.

In an embodiment, the protein has a molecular weight of from about 10³ Daltons to about 10¹² Daltons, e.g. from about 10³ Daltons to about 10⁶ Daltons.

The protein that is detected may be a membrane protein or a soluble protein and is preferably a membrane protein.

Membrane proteins can be grouped into integral membrane proteins and peripheral membrane proteins. Integral membrane proteins may have one or more segments embedded within a membrane and may be bound to the lipid bilayer. Peripheral membrane proteins may be temporarily associated with the lipid bilayer and/or integral membrane proteins. In an embodiment, the membrane protein is an integral membrane protein.

In an embodiment, the membrane protein is an integral membrane protein selected from G protein-coupled receptors (GPCRs), membrane transporters, membrane channels, ATP-binding cassette transporters (ABC-transporters), proton driven transporters, solute carriers, and outer membrane proteins (OMPs). In specific examples, the membrane protein is selected from aquaporin Z (AqpZ); ammonia channel (AmtB), translocator protein (TSPO), large-conductance mechanosensitive channel (MsCl), multidrug efflux pump subunit (AcrB), and ATP-binding cassette transporter (BtuCD).

The membrane protein may be a bacterial membrane protein, such as a membrane protein from E. coli, e.g. an inner membrane protein. The way lipids interact and modify the structure and function of bacterial membrane proteins is of great interest, since this can impact the efficacy of antibiotics.

Soluble proteins are present outside of a cellular membrane in organisms, e.g. in the cytoplasm.

The solution comprising a hybrid detergent and a protein may be provided by known methods. Details about membrane protein expression, membrane protein purification, and tuning advices for different mass spectrometers are all available in the literature (e.g. Laganowsky et al., Nat. Protoc., 2013, 8, 639-651; Laganowsky et al., Nature, 2014, 510, 172-175; Gault et al., Nat. Methods, 2016, 13, 333-336). Proteins may also be commercially available. To produce high quality mass spectra, the protein should preferably be relatively pure and homogenous, equivalent to crystallographic-grade material. The solution comprising a hybrid detergent and a protein may also be provided using the method of preparing a protein sample that is described in greater detail below.

The protein may be in the form of a complex with a ligand. The present methods may therefore be used to detect binding between a protein and a ligand. In particular, a method of the present invention may allow one or more structural characteristics (e.g. stoichiometry) of a protein-ligand complex to be determined, and/or may also be used to detect conformational changes that take place upon binding of a therapeutic agent to the protein.

Binding of the ligand to the protein may be via a non-covalent or a covalent interaction, though will typically be via a non-covalent interaction. In particular, binding of the ligand to the protein may be via intermolecular forces such as ionic bonds, hydrogen bonds and van der Waals forces. Binding of the ligand to the protein may be reversible or irreversible. In an embodiment, the ligand is bound to the protein via a reversible bond.

Ligands with which the protein may be in the form of a complex include one or more of therapeutic agents, lipids, nucleotides and nucleosides.

In preferred embodiments, the protein may be in the form of a complex with one or more lipids. Membrane proteins are more likely to be detected in the form of a complex with one or more lipids due to the hydrophobic regions on their surface, as well as the “native” membrane environment from which they are obtained. The hybrid detergents of the present invention are particularly suited to methods in which the lipidation of a membrane protein is explored.

Examples of lipids include, but are not limited to, fatty acids, glycerolipids, glycerophospholipids, sphingolipids, sterol lipids, prenol lipids, saccharolipids and polyketides. Particularly preferred lipids with which the protein may be in the form of a complex include lipopolysaccharides (e.g. lipid A and Kdo2-lipid A (KLA)) and phospholipids (e.g. phosphatidylglycerol (PG), phosphatidylethanolamine (PE), and cardiolipin (CDL) such as PG (14:0), PE (14:0) and CDL (14:0))).

In one embodiment, the protein is in the form of a complex with one or more therapeutic agents. These embodiments are preferred when the protein is a membrane protein, since these proteins are key targets for therapeutic agents. The therapeutic agent may be an active compound which, when administered to an organism (human or non-human animal), induces a desired pharmacologic, immunogenic, and/or physiologic effect by local and/or systemic action. Examples of therapeutic agents include, without limitation, drugs, vaccines and biopharmaceutical agents. Thus, therapeutic agents may include small molecule drugs, therapeutic proteins, peptides and fragments thereof (whether naturally occurring, chemically synthesised or recombinantly produced), and nucleic acid molecules (including both double- and single-stranded molecules, gene constructs, expression vectors, antisense molecules and the like). Therapeutic agents may also include substrates, inhibitors, activators, neurotransmitters, agonists and antagonists. The therapeutic agent may be a synthetic or naturally occurring compound. The therapeutic agent may be a drug candidate or other agent suspected of having therapeutic application.

Particular examples of therapeutic agents include, but are not limited to, anti-cancer agents, anti-infective agents (e.g. antibiotics and antiviral agents), analgesic agents, anorexic agents, anti-inflammatory agents, antiepileptic agents, anaesthetic agents, hypnotic agents, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics agents, hormones, nutrients, antiarthritics agents, antiasthmatic agents, anticonvulsants, antihistamines, antinauseants agents, antineoplastic agents, antipruritics agents, antipyretic agents; antispasmodic agents, cardiovascular agents (e.g. calcium channel blockers, beta-blockers, beta-agonists, antiarrhythmic agents, antihypertensive agents, diuretics and vasodilators), central nervous system stimulants; decongestants, hormones, bone growth stimulants, bone resorption inhibitors, immunosuppressive agents, muscle relaxants, psychostimulants, sedatives and tranquilisers. It will be appreciated that this list of therapeutic agents is merely illustrative and should not be considered to be limiting. Many other therapeutic agents are known in the art and may be utilised in a method of the present invention. A detailed description of various therapeutic agents may be found in e.g. Remington's Pharmaceutical Sciences (21st edition, 2005, Mack Publishing Company). The therapeutic agent may exhibit optical isomerism and/or diastereoisomerism. Accordingly, the therapeutic agent may be in the form of a single enantiomer or diastereoisomer, or a mixture (e.g. a racemic mixture) thereof.

In an embodiment, the therapeutic agent has a molecular weight of less than 2000 Daltons, e.g. less than 1500 Daltons, e.g. less than 1000 Daltons, e.g. less than 500 Daltons. In an embodiment, the therapeutic agent is a non-polymeric organic compound having a molecular weight of less than 1000 Daltons, e.g. less than 800 Daltons, e.g. less than 500 Daltons.

In an embodiment, the therapeutic agent is an inhibitor or an activator, e.g. an activator or inhibitor of the protein to which it is bound. In an embodiment, the therapeutic agent is an antibiotic.

A method of the present invention may allow therapeutic agents to be screened. In contrast to indirect methods such as fluorescence or calorimetry, the present method may allow therapeutic agents to be screened directly. In particular, a method may be used to screen for the binding of activators and transporter substrates which are difficult to screen using conventional in vivo methodologies. Moreover, unlike X-ray crystallography, the present methods are not complicated by the inherent structural flexibility of protein-therapeutic agent complexes and may allow the dynamical behaviour of proteins and their interaction with therapeutic agents to be studied.

The protein may be in the form of a complex with more than one ligand. Thus, for instance, a method of the present invention may be used to determine whether the presence of a first ligand affects binding of a second ligand to the protein, in particular whether the presence of a lipid affects binding of a second ligand, such as a therapeutic agent, to the protein. For instance, a method of the present invention may be used to determine whether a lipid affects binding of an antibiotic to a bacterial membrane protein.

The present invention involves the use of a solution in which the hybrid detergent and protein are contained. The hybrid detergent is preferably associated with the protein so that the hybrid detergent may stabilise the protein in the gas phase.

In preferred embodiments, particularly where a membrane protein is used, the solution comprises a detergent aggregate in which the protein is contained, the detergent aggregate being formed by the hybrid detergent. The detergent aggregate is preferably in the form of a micelle (e.g. a substantially spherical micelle or a worm-like micelle), but may also be in the form of a vesicle or a tubular aggregate.

Where the protein is encapsulated in a detergent aggregate for solubilisation, the aggregate is believed to shield the protein at least partially during the electrospray ionisation process. Without wishing to be bound by theory, it is believed that the aggregate may shield the protein during the droplet phase of the electrospray ionisation process and, moreover, may afford at least partial shielding from ionisation of the protein during this process. The detergent aggregate may exert a pressure sufficient to maintain the structure of the protein, thereby minimising the deleterious effects associated with vaporisation and substantially retaining interactions between the protein and any ligand as well as interactions within any subunits of the protein.

The solution will typically comprise a plurality of detergent aggregates containing the protein. The solution may be formed by e.g. incubating the protein in the presence of the detergent.

Preferably, the protein is maintained in the solution in an intact, folded state. This may allow the protein to be detected in its folded, i.e. “native”, state. Alternatively, the protein may be present in the solution in a partially folded or unfolded state.

The solution may also contain one or more detergents in addition to the hybrid detergent. Examples of other detergents include non-ionic detergents such as n-dodecyl-D-maltoside, nonylglucoside, glycosides, neopentyl glycols, facade EM, maltosides, glucosides, and mixtures thereof. However, it is generally preferred for the solution to be substantially free from detergents other than the hybrid detergent.

In an embodiment, the hybrid detergent is present in the solution at a concentration of from about 100 UM to about 100 mM, e.g. from about 200 UM to about 1 mM. In an embodiment, the protein is present in the solution at a concentration of from about 10 nM to about 1 mM, e.g. from about 1 μM to about 100 UM.

In an embodiment, the molar ratio of the hybrid detergent to the protein is from about 0.5:1 to about 10,000:1. Where the protein is a soluble protein, then the hybrid detergent is not required to solubilise the protein in the aqueous mass spectrometry environment, and so the hybrid detergent may be used in lower amount, e.g. from about 0.5:1 to about 50:1, e.g. from about 0.75:1 to about 10:1, and more preferably from about 1:1 to about 5:1.

However, where the protein is a membrane protein, larger molar ratios of hybrid detergent to protein are preferred, e.g. from 50:1 to 10,000:1, e.g. from about 100:1 to about 5,000:1, e.g. from about 200:1 to about 1,000:1. In a preferred embodiment, the molar ratio of the detergent to the membrane protein is less than or equal to 1,000:1.

To minimise dissociation of the protein and/or precipitation of the protein, the hybrid detergent is preferably present in the solution at a concentration at least equal to the critical aggregation concentration (CAC) of the detergent.

In preferred instances, the hybrid detergent may be present in the solution at a concentration of at least 1.5 times, and preferably at least 1.75 times, the CAC of the detergent. The hybrid detergent may be present in the solution at a concentration of up to 3 times, and preferably up to 2.5 times, the CAC of the detergent. Thus, the hybrid detergent may be present in the solution at a concentration of from 1.5 to 3 times, and preferably from 1.75 to 2.5 times, e.g. 2 times, the CAC of the detergent. These concentrations are particularly suitable for the gradual delipidation of phospholipids from proteins using different hybrid detergents.

In other instances, the hybrid detergent may be present in the solution at a concentration of at least 35 times, and preferably at least 45 times, the CAC of the detergent. The hybrid detergent may be present in the solution at a concentration of up to 65 times, and preferably up to 55 times, the CAC of the detergent. Thus, the hybrid detergent may be present in the solution at a concentration of from 35 to 65 times, and preferably from 45 to 55 times, e.g. 50 times, the CAC of the detergent. These concentrations are particularly suitable for delipidation of lipopolysaccharides from proteins.

The CAC of the hybrid detergent may be determined experimentally, e.g. using a dynamic light scattering method. A suitable method is described in the experimental section herein.

Where the protein is in the form of a complex with a therapeutic agent, the micellar solution preferably comprises a molar excess of the therapeutic agent as compared to the protein. In an embodiment, the molar ratio of the therapeutic agent to the protein is at least 2:1, e.g. at least 5:1, e.g. at least 10:1. In an embodiment, the therapeutic agent is present in the solution at a concentration of at least 100 nM, e.g. from 1 μM to 500 μM.

Other ligands such as lipids, nucleotides and nucleosides may be in a complex with the protein in its native environment and, as such, will typically not be added to the solution.

The solution may comprise one or more other components. In particular, the solution preferably contains a buffer. Ammonium acetate is particularly preferred in this regard. The concentration of ammonium acetate is preferably at least 150 millimolar. Preferably the pH of the buffer is in the range of from about 5 to about 8.

Buffer exchange and concentration of the solution may be achieved using suitable techniques and devices known in the art, e.g. using a Micro Bio-Spin® column (Bio-Rad Laboratories) or a Vivaspin device (GE Healthcare).

The protein is detected using a mass spectrometer comprising a nanoelectrospray ionisation source, a mass analyser, and a detector. The mass spectrometer is preferably adapted to transmit and detect ions having mass-to-charge (m/z) ratios in the range of e.g. from about 100 m/z to about 32,000 m/z. Preferably, the mass spectrometer is operated under conditions suitable for maintaining and focusing large macromolecular ions. By way of illustration, and without limitation, the mass spectrometer is preferably, an orbitrap mass spectrometer, such as a Q-Exactive hybrid quadrupole-orbitrap mass spectrometer. The resolution provided by such instruments is particularly suited to resolving peaks generated from a complex comprising a protein bound to a ligand.

The nanoelectrospray ionisation source is used to vaporise the solution. Nanoelectrospray ionisation is a technique well known in the art (see e.g. Wilm et al, Anal. Chem. 1996, 68, 1-8; and Wilm et al, Int. J. of Mass Spec. and Ion Proc. 1994, 132, 167-180). The use of nanoelectrospray ionisation allows ions, and in particular highly charged ions, to be generated directly from solution. The formation of highly charged ions may allow the detection of high mass complexes at relatively low mass-to-charge (m/z) ratios. The use of a nanoelectrospray ionisation is also desirable from the point of view of allowing a protein complex, or subunits of a complex, to remain substantially intact. In performing a method of the present invention, it may be preferable to use a nanoflow capillary, e.g. a gold-coated nanoflow capillary, to vaporise the solution.

The solution is preferably vaporised under conditions such that the hybrid detergent is dissociated from the protein. For instance, where the solution comprises detergent aggregates in which the protein is contained, the solution is preferably vaporised under conditions such that the protein is released from the aggregate. It may, however, occasionally be useful to detect a protein bound to one or more hybrid detergent molecules, e.g. in order to study dissociation of the detergent from the protein. The hybrid detergent may be dissociated from the protein by means of collisions between gas molecules and protein-detergent complexes which increase the internal energy of the protein-detergent complex and lead to its dissociation. Preferably, the vaporisation conditions are selected so that the protein is detected substantially intact. Preferably, the conditions inside the mass spectrometer are selected to rapidly remove the hybrid detergent from the protein.

Ionisation of the protein may occur during the step of vaporising and/or after release of the protein from the detergent. In some instances, portions of the protein, e.g. hydrophilic/cytoplasmic domains, may become ionised prior to release of the protein from the detergent. Typically, ionisation of the protein occurs during and/or after dissociation of the hybrid detergent from the protein.

In an embodiment, release and/or ionisation of the protein occurs in a collision cell present within the mass spectrometer. Release and/or ionisation of the protein may be achieved by adjusting acceleration voltages and/or pressures within the collision cell to remove the detergent while retaining the peaks of the protein.

Mass spectrometer parameters may be optimised for maximal desolvation and detergent removal, while minimising protein activation. In particular, one or more of the following parameters may be optimised: collision voltage, in-source trapping voltage, collision gas pressure, collision gas type, and source pressure. Optimisation of parameters may be achieved by first setting the instrument parameters to relatively high activation settings for proteins. Then iteratively, each of the aforementioned five parameters may be adjusted to produce resolved mass spectra while minimizing over-activation of the target protein.

In preferred embodiments, a mass spectrometer, e.g. an orbitrap mass spectrometer such as a Q-Exactive hybrid quadrupole-orbitrap mass spectrometer (e.g. available from Thermo Scientific), is operated under one or more of the following conditions: (i) an injection flatapole voltage of about 2.0 to about 8.0 V, e.g. about 4.0 to about 8.0 V, e.g. about 6.0 to about 8.0 V. e.g. 7.9 V; (ii) an inter flatapole lens voltage of about 2.0 to about 7.0 V, e.g. about 4.0 to about 7.0 V, e.g. about 6.0 to about 7.0 V, e.g. 6.9 V; (iii) a bent flatapole voltage of about 2.0 to about 6.0 V, e.g. about 4.0 to about 6.0 V, e.g. about 5.0 to about 6.0 V, e.g. 5.9 V; (iv) a transfer multipole of about 3.0 to about 5.0 V, e.g. about 3.25 to about 4.75 V, e.g. about 3.5 to about 4.5 V, e.g. 4 V; (v) an acceleration voltage in the higher-energy collisional dissociation (HCD) cell of from about 0 to about 250 V, e.g. from about 50 to about 225 V, e.g. from about 100 to about 200 V; (vi) an in-source trapping activation of about 0 V to about 300 V; and (vii) a pressure in the HCD cell of from about 8.0×10⁻¹⁰ to about 2.0×10⁻⁹ mBar, e.g. from about 8.5×10⁻¹⁰ to about 1.5×10⁻⁹ mBar, e.g. from about 9×10⁻¹⁰ to about 1×10⁻⁹ mBar.

The Q-Exactive mass spectrometer may also be operated under one or more of the following conditions: (viii) transient time of about 20 to about 150 ms, e.g. about 50 to about 125 ms, e.g. about 100 ms; (ix) a noise level parameter of about 2.5 to about 5, e.g. about 3 to about 4, e.g. about 3 to about 3.5, e.g. 3; (x) resolution of about 8,000 to about 140,000, e.g. about 10,000 to about 100,000, e.g. about 15,000 to about 30,000, e.g. about 17,500.

The Q-Exactive mass spectrometer may also be operated under one or more of the following conditions: (xi) a capillary voltage of from about 0.8 to about 2.2 kV, e.g. from about 1.0 to about 2.0 KV, e.g. from about 1.2 to about 1.8 kV, e.g. 1.2 V; (xii) a source temperature of from about 25 to about 100° C., e.g. from about 50 to about 100° C., e.g. about 100° C.; (xiii) a DC voltage in the transfer multipole of from about 2 to about 4 V, e.g. from about 3 to about 4 V; (xiv) a voltage in the C-trap entrance lens of from about 0 to about 7 V, e.g. from about 2 to about 4 V, e.g. from about 5 to about 6 V, e.g. 5.8 V.

It will be appreciated that any of conditions (i) to (xi) may be applied alone or in combination with one or more, or all, of the other conditions.

Preferably, minimal activation energy is used to dissociate the protein from the detergent. In an embodiment, the laboratory frame energy is from about 500 to about 5000 electron volts, e.g. from about 500 to about 1500 electron volts. The term “laboratory frame energy” as used herein refers to the collision voltage multiplied by charge state of the protein.

It will be appreciated that the values described above represent the magnitude of the settings on the mass spectrometer. The values themselves may be positive or negative, depending on whether the mass spectrometer is operated in positive or negative mode. Typically, the mass spectrometer will be operated in positive polarity. A person of skill in the art will understand which values are negative and which values are positive in each mode.

The ionised protein is then resolved and detected and, if desired, further characterised. In particular, in embodiments in which the protein is in the form of a complex with a ligand such as a lipid, ions in which the ligand is bound to the protein or a fragment thereof can be detected directly using the mass spectrometer, rather than inferred indirectly from mass spectra of the separate components (ligand and protein). Moreover, where the solution or the complex comprises more than one ligand, the binding of one or more of said components to the protein may be detected simultaneously. Thus, the present methods may be used to detect concomitant binding of the protein e.g. with a lipid and one or more other species. The binding of one or more hybrid detergent molecules to the protein, optionally concomitantly with one or more ligands, may also be directly detected though this is less preferred.

In some embodiments, the method comprises detecting the membrane protein in the form of a complex with a ligand, such as a lipid, in a form in which it is substantially intact, and further interrogating the membrane protein-ligand complex by stripping the ligand from the membrane protein, fragmenting the ligand to give ligand fragments, and detecting the ligand fragments. This can be achieved using a mass spectrometry instrument, such as mentioned above, in which an ion peak may be selected for further fragmentation e.g. using higher-energy collisional dissociation (HCD). This “top-down” approach is a highly effective way of carrying out the method of the present invention.

It will be appreciated that the fragmented ligand is in an ionised form in the mass spectrometer and, as with all mass spectrometry species, is resolved using a mass analyser before it being detected using a detector.

The detected fragments of ligand may be used to verify the nature of ligand in the membrane protein-ligand complex.

### Membrane Protein Sample Preparation Methods

The hybrid detergents described herein may be in methods of preparing a protein sample. These methods comprise: (i) providing a solution which comprises an extraction detergent aggregate in which a protein is contained; and (ii) contacting the extraction detergent aggregate with a hybrid detergent to give a solution which comprises a hybrid detergent aggregate in which the protein is contained.

In some embodiments, the method comprises extracting the protein from its native membrane by contacting the protein with an extraction detergent to form the solution comprising the extraction detergent aggregate. It is well-understood in the art that native membranes are those membranes in an organism in which the membrane protein is present.

A membrane protein may be provided in its native environment by expressing the membrane protein in an organism. In embodiments, the method may comprise overexpression a membrane protein in an organism, for instance by introducing gene vectors for overexpression of the membrane protein into the organism. Typically, bacteria such as E. coli will be used. Mammalian cell lines (e.g. 293T), insect cells and yeast may also be used. Methods in which membrane proteins are overexpressed are known in the art and are described e.g. in Laganowsky et al., Nat. Protoc. 2013, 8, 639-651 (see also Drew et al., Nat. Protoc. 2008, 3, 784-798).

Following expression of the membrane proteins, the cells may be collected, e.g. by centrifugation. The cells may be then lysed, e.g. using a lysis buffer, to provide a lysate.

Suitable lysis buffers may comprise tris(hydroxymethyl)aminomethane (‘Tris’, e.g. about 20 mM). Other components in the lysis buffer may include NaCl (e.g. about 300 mM). The pH of the lysis buffer may be from about 7 to about 8, e.g. 7.4. In preferred embodiments, then lysate will be homogenised, e.g. by being passed through a microfluidizer, and insoluble material removed by centrifugation.

The lysed membranes may be suspended in a buffer. Suitable buffers include Tris (e.g. about 20 mM). Other components in the buffer may include NaCl (e.g. about 100 mM) and/or glycerol (e.g. 0.2 v/v). The suspension may be homogenised, e.g. using a pestle and glass tube.

Once the protein has been provided in its native membrane, it is contacted with an extraction detergent. The extraction detergent extracts the membrane protein from its native membrane and forms a detergent aggregate in which the membrane protein is contained. Preferably, the membrane protein is present in the extraction detergent aggregate in a lipidated form. This means that the membrane protein can be delipidated on contact with a hybrid detergent in step (ii), the degree of delipidation varying between hybrid detergents.

The extraction detergent is preferably a detergent that exhibit weak delipidating properties, such as n-dodecyl-ß-D-maltoside (DDM).

Detergent exchange step (ii) may be carried out using a number of different methods. Preferably, detergent exchange is carried out using size exclusion chromatography (SEC), though other methods such as immobilised metal ion affinity chromatography (IMAC) may also be used.

The hybrid detergent is preferably contacted with the extraction detergent aggregate to give an aqueous solution containing the hybrid detergent at a CAC concentration as described above in the section on mass spectrometry. The concentration of hybrid detergent and protein in the solution produced in step (ii) are preferably also as described above in the section on mass spectrometry.

The solution prepared in step (ii) may be used in mass spectrometry methods, e.g. such as those described above. However, the solution may also be used in other protein analysis methods such as cryogenic transmission electron microscopy (cryo-TEM), nuclear magnetic resonance (NMR), x-ray crystallography (XRC), small-angle neutron scattering (SANS), dynamic light scattering (DLS), size-exclusion chromatography coupled to multi-angle light scattering (SEC-MALS), and circular dichroism (CD) spectroscopy. Thus, the present invention provides method for analysing proteins, in particular membrane proteins, in which the solution prepared in step (ii) is used.

The hybrid detergents described herein may also be used for extracting a protein directly from its native membrane. Thus, the present invention provides a method of extracting a membrane protein from its native membrane, wherein the method comprises:
i. providing a protein in its native membrane; and
ii. contacting the protein with a hybrid detergent;
   wherein the detergent forms a detergent aggregate in which the membrane protein is contained.

### Lipidome Interrogation Methods

The present invention is based, at least in part, on the discovery that the hybrid detergents described herein may be used to interrogate the lipidome of a protein, typically a membrane protein. In particular, it has been discovered that the degree of phospholipid delipidation is correlated with the HLB and/or p values of a detergent, such as the hybrid detergents described herein. In contrast, the degree of lipopolysaccharide delipidation is independent from the HLB and/or p values.

Thus, the present invention provides a method of interrogating the lipidome of a protein of interest, said method comprising:
providing at least three solutions comprising a detergent and the protein, a different detergent being used in each solution;
providing a mass spectrometer comprising a nanoelectrospray ionisation source, a mass analyser, and a detector, and for each of the solutions:
   vaporising the solution using the nanoelectrospray ionisation source;
   ionising the protein;
   resolving the ionised protein using the mass analyser;
   detecting the resolved protein using the detector; and
   determining the degree of lipidation in the detected protein;
calculating at least one of the HLB and the p value of each of the detergents; and
correlating the HLB and/or p value of the detergents with the degree of lipidation in the detected protein.

Preferably, at least one, e.g. at least two, of the solutions comprise a hybrid detergent as defined herein. In preferred embodiments, the method is carried out using the detergents from a detergent library (kit) described above.

The mass spectrometry method is preferably carried out as described above in the section on mass spectrometry methods.

The interrogation method may comprise detecting the protein in a lipidated state in at least one, and preferably at least two, and more preferably all, of the at least three solutions.

If a correlation is observed between the HLB and/or p value of the detergent and the degree of lipidation in the detected protein, this implies that the protein is lipidated with phospholipids, e.g. at least one of phosphatidylglycerol (PG), phosphatidylethanolamine (PE), and cardiolipin (CDL). If there is no correlation between the HLB and/or p value of the detergent and the degree of lipidation in the detected protein, this implies that the protein is lipidated with a lipopolysaccharide.

### EXAMPLES

The following non-limiting examples illustrate the present invention.

### Methods

The following methods were employed in the experiments of the examples.

### Preparation of Hybrid Detergents

Building block synthesis: Apart from the perbenzylated glucose derivative (BN-Glu-OH), all building blocks were purchased and used as supplied. The perbenzylated glucose derivative was synthesized using the following procedure:

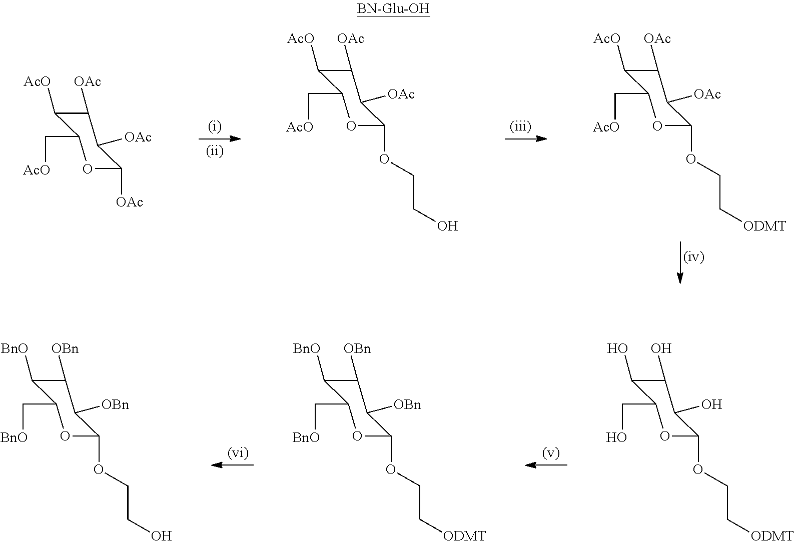

Reaction conditions used for the individual steps were as follows: (i) Bn-E1, BF₃ (OEt)₂, DCM, −10° C. to RT, 16 h; (ii) H² (1 bar), Pd/C (cat.), MeOH, RT, 24 h; (iii) 4,4′-dimethoxytrityl chloride, NEt₃, toluene/DCM (v:v, 4:1), RT, 24 h; (iv) NaOMe (cat.), MeOH, RT, 20 h; (v) NaH (60 w %), benzyl bromide, DMF, 50° C., 20 h; (vi) HCl (37 w %), MeOH/DCM (v:v, 16:1), RT, 20 h.

Head group synthesis: The starting materials *H¹—OH and *H²—OH [where * represents protected versions of the head groups H¹ and H² that are present in the final detergents] were dissolved in equimolar amounts in dry THF (250 or 300 mL). NaH (60 w %, 3×H¹—OH molar amount) and catalytic amounts of 15-crown-5 were added and the mixture was stirred at 50° C. for 1 h. Subsequently, MDC (1×H¹—OH molar amount), catalytic amounts of 18-crown-6, and catalytic amounts of potassium iodide were added. The mixture was stirred at 80° C. for 25 h. The mixture was allowed to cool down to RT and H₂O (5 or 10 mL) was added. Solvent was removed under reduced pressure. The remaining material was suspended with a mixture of Brine (150 or 200 mL), H₂O (150 or 200 mL), and DCM (300 or 400 mL). The aqueous layer was extracted with DCM (3×300 or 5×200 or 8×175 mL). The combined organic layers were dried over Na₂SO₄, solids were filtered off, and solvent was removed under reduced pressure. Column chromatography (SiO₂, DCM/EtOAc, 1:0→1:1 or 4:1+4% or 10% MeOH) was used to separate the products, and their identity was confirmed using 1H NMR, 13C NMR and MS(ESI+).

| Building blocks | | Head group product | | |
|---|---|---|---|---|
| *H¹—OH | *H²—OH | *H¹*H¹ | *H¹*H² | *H²*H² |
| [pG0]-OH | Bn-E4 | 4% | 51% | 22% |
| (17.8 mmol) | | | | |
| Bn-Glu-OH | Bn-E4 | 18% | 42% | 13% |
| (10.3 mmol) | | | | |
| Bn-E1 | Bn-E4 | 27% | 37% | 19% |
| (17.6 mmol) | | | | |

Detergent synthesis method 1 (mixed detergent synthesis):

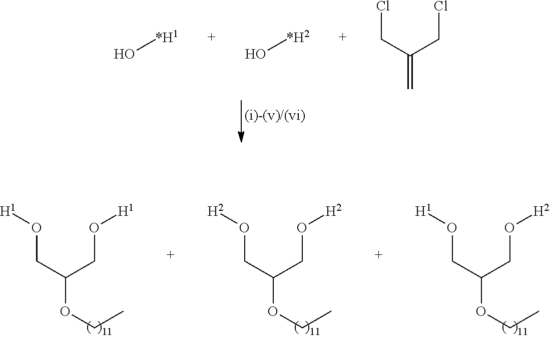

Reaction conditions were as follows: (i) NaH (60 w %), THF, 50 to 80° C., 17 or 23 h; (ii) O3, DCM/MeOH (v:v, 1:1), −78° C., 1 h; (iii) NaBH₄, −78° C. to RT, 16 h; (iv) NaH (60 w %), 1-bromododecane, DMF, 50° C. to RT, 17 h; (v) H₂(5 bar), Pd/C (cat.), MeOH, RT, 19 h; (vi) HCl (37 w %), MeOH, RT, 23 or 74 h. When detergents 1, 3 and 4 were synthesized, a further step was applied: (vi) HCl (37 w %), MeOH, RT, 23 h. When detergents 4, 5 and 6 were synthesized, the method consisted of just steps (i)-(v).

Detergent synthesis method 2 (single detergent synthesis):

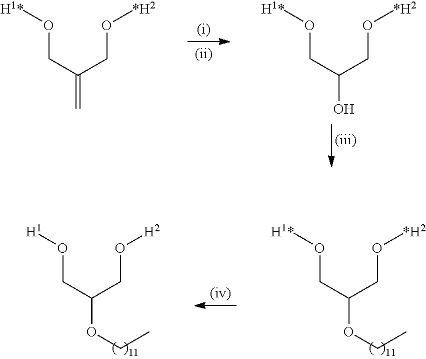

Reaction conditions were as follows: (i) O3, DCM/MeOH (v:v, 1:1), −78° C., 1 h; (ii) NaBH₄, −78° C. to RT, 16 h; (iii) 1-bromododecane, NaH (60 w %), DMF, 50° C. to RT, between 17 and 22 h. Removal of the protecting group was carried out as follows. For detergent 1: (iv) HCl (37 w %), MeOH, RT, 2×12 h. For detergents 2, 4, 5 and 6: (iv) H₂ (1 or 5 bar), Pd/C (cat.), MeOH/THF (v:v, 10:1), RT, 24 or 48 h. For detergent 3: (iv) Hz (5 bar), Pd/C (cat.), MeOH, RT, 15 h; (v) HCl (37 w %), RT, 12 h.

### Determining the Packing Parameter of the Detergents

To calculate the packing parameter of the detergents, it is necessary to determine Vₜₐᵢₗ (the volume of the hydrophobic tail), Iₜₐᵢₗ (the length of the hydrophobic tail), and A_{head} (the area of the head group that is occupied at the interface between the detergent aggregate and solvent).

To determine A_{head}, the three-dimensional structure of detergent head groups (without tail) was modelled using a MM2 force field as implemented in the software ChemBio3D v14.0 (PerkinElmer) and calculated their collision cross sections (CCSs) using a projection approximation algorithm (von Helden, J. Phys. Chem., 1993, 97, 8182-8192). The CCS of a detergent head group was taken as A_{head}. Vₜₐᵢₗ was calculated using the van der Waals volume calculation method (Zhao et al., J. Org. Chem., 2003, 68, 7368-7373) and Iₜₐᵢₗ was calculated using ChemBio3D v14.0 (PerkinElmer).

### Protein Expression and Membrane Preparation

Membrane protein expression was performed as previously described (Laganowsky et al., Nature, 2014, 510, 172-175; Urner et al., Nat. Commun., 2020, 11, 1-10). Plasmids were transformed into C43 (DE3) cells by mixing 1 μL of plasmid solution (plasmid concentration=100 ng/μL) with a 50 μL aliquot of C43 cells (purchased from Cambridge Bioscience). The cells were incubated on ice for 30 min, heat shocked at 42° C. for 45 s, and cooled on ice for 2 min. LB Broth (450 μL of a 25 g/L aqueous solution) was added. The mixture was shaken with 180 rpm at 37° C. for 1 h. One 50 μL aliquot of this mixture was plated on an agar plate (agar medium composition: 25 g/L LB Broth and 15 g/L agar in water, supplemented with 100 μg/mL ampicillin). The plate was stored overnight at 37° C.

Up to five colonies were picked and transferred into starter culture medium (5 ml of 25 g/L LB Broth, supplemented with 100 μg/mL of ampicillin). The mixture was shaken with 180 rpm at 37° C. for 8 h. The starter culture was transferred into overnight culture medium (400 mL of 25 g/L LB Broth, supplemented with 100 μg/mL of ampicillin). The mixture was shaken with 180 rpm for 15 h at 37° C. The overnight culture was transferred into in 12 L expression medium (12×1 L of 25 g/L LB Broth, supplemented with 100 g/ml of ampicillin). Cells were shaken with 180 rpm at 37° C. until an optical density value at 600 nm (OD600) between 0.7 and 1.0 was reached.

Protein expression was induced by adding isopropyl-ß-D-thiogalactopyranoside (12×1 mL of a 0.5 M aqueous solution) and the cells were shaken with 180 rpm at 37° C. for another four hours. Cells from a 12 L expression batch were harvested by centrifugation (5,000×g, 10 min), suspended in 100 mL buffer (20 mM Tris, 300 mM NaCl, 20% v/v glycerol, pH=7.4, supplemented with two protease inhibitor tablets), and lysed using a Microfluidizer. After supernatant clarification (20,000×g, 20 min, 4° C.), the membranes were pelleted down (100,000×g, 2 h, 4)° C. and homogenized in 6 mL buffer B (20 mM Tris, 100 mM NaCl, 20% v/v glycerol, pH=7.4, supplemented with one protease inhibitor per 50 mL). The membrane suspension was separated into 2 mL aliquots, which were frozen in liquid nitrogen and could be stored at −80° C. for up to two years.

### Membrane Protein Purification

A membrane aliquot (2 mL) was added to a mixture of 9 mL buffer B and 1 mL DDM stock solution (10 w % DDM in MilliQ water). The suspension was agitated for one hour at a temperature of 4° C. before the supernatant was clarified by centrifugation (4,000×g, 30 min, 4)° C.). The supernatant was purified by IMAC as described below.

First, the protein-containing supernatant was mixed with the IMAC resin as follows: 7 mL of nickel-nitrilotriacetic acid (Ni-NTA) agarose suspension (50%, Quiagen) were mixed with 23 mL of MilliQ. The supernatant was clarified by centrifugation (4,000×g, 2 min, 4° C.), discarded, and the procedure was repeated. The ethanol-free resin was suspended in 5 mL IMAC wash buffer (50 mM Tris, 200 mM NaCl, 20 mM imidazole, 10% v/v glycerol, 2×critical aggregation concentration of DDM, pH=8) and the protein-containing supernatant was added. The mixture was agitated at 4° C. for 15 minutes and then loaded into an empty gravity flow column (14 cm high, 1.5×1.2 cm polypropylene columns from Bio-Rad). Once the liquid passed though the column, the IMAC resin was washed with 45 mL IMAC wash buffer, 45 mL of a IMAC wash/elute buffer mixture (9/1 v/v, 2×critical aggregation concentration of DDM, pH=8), and the protein was eluted with 10 mL IMAC elute buffer (50 mM Tris, 200 mM NaCl, 250 mM imidazole, 10% v/v glycerol, 2×critical aggregation concentration of DDM, pH=8).

The eluted membrane protein was concentrated with centrifugal filters (Amicon®). The molecular weight cut-off (MWCO) of the centrifugal filters was adjusted to the molecular weight of the proteomicelle formed with the expected membrane protein oligomer (MWCO=50 kDa for TSPO, MsCl-GFP; MWCO=100 kDa for AqpZ GFP, AmtB-MBP, AcrB, BtuCD).

The volume was reduced to 5 mL and His-tagged Tobacco Etch Virus (TEV) protease was added to GFP- or MBP-tagged membrane proteins (1-2 mg TEV). The mixture was transferred into dialysis cassettes (MWCO=3.5 kDa) and dialyzed for 16 hours at 4° C. against dialysis buffer (50 mM Tris, 200 mM NaCl, 20 mM imidazole, 10% v/v glycerol, 2×critical aggregation concentration of DDM, 5 mM 2-mercaptoethanol, pH=8). Purely His-tagged membrane proteins were dialyzed under similar conditions just without the use of TEV.

The dialyzed protein mixture was then purified by reverse IMAC. For this purpose, the IMAC column was washed with 20 mL dialysis buffer. The dialyzed protein mixture was passed over the column and the flow-though was collected. The column was washed with another 5 mL of dialysis buffer and the flow-through was collected. The combined flow-thoughts were concentrated in centrifugal filters until a protein concentration between 30 UM and 50 UM was reached. The protein solutions were separated into 45 μL aliquots, frozen in liquid nitrogen, and could be stored at −80° C. for up to one year.

### General Delipidation of Membrane Proteins

Delipidation was carried out with a detergent exchange from DDM to the detergent of interest (i.e. OG, C8E4, 1, 2, 3, 4, or 5) over a 3 mL SEC column (Superdex 200 10/300GL column, product number: 17-5175-01). The column was stored in a mixture of ethanol and MilliQ water (¼, v/v) and was equilibrated with an Äkta setup that was operated at 4° C. in a cold room. The Äkta was equipped with a sample fractionator and the chromatogram was monitored with a UV/VIS detector. The column was washed with 1.2 column volumes (CVs) of MilliQ water, 1 CV of an aqueous sodium hydroxide solution (0.5 mM), and 1.2 CVs of MilliQ water before it was equilibrated with 1.2 CVs of ammonium acetate solution (200 mM, pH=6.8, 2×critical aggregation concentration of the detergent of interest). A 45 μL aliquot of purified protein was used for each detergent exchange. The protein was eluted over 1.5 CVs of detergent-containing ammonium acetate solution at a flow rate of 0.2 mL/min. The main protein-containing fractions were combined and concentrated using Amicon® Ultra 0.5 mL centrifugal filters to a final volume of about 20 to 30 μL.

### Lipopolysaccharide Delipidation at High Detergent Concentration

To achieve lipopolysaccharise (LPS) delipidation, IMAC resin was prepared as follows: an empty bio-spin column (Bio-Rad) was loaded with 500 μL of Ni-NTA agarose suspension (50%, Quiagen). The resin was washed with 500 μL MilliQ water, 500 μL IMAC elute buffer (50 mM Tris, 200 mM NaCl, 250 mM imidazole, 10% v/v glycerol, 1 w % of detergent 1 or hybrid detergent 3, pH=8), and 500 μL IMAC wash buffer (50 mM Tris, 200 mM NaCl, 20 mM imidazole, 10% v/v glycerol, 1 w % of detergent 1 or hybrid detergent 3, pH=8). The column was loaded with two 45 μL aliquots of His-tagged membrane protein (see above for details about membrane protein preparation).

To identify how many CVs of IMAC wash buffer are needed to delipidate LPS, six columns were prepared in parallel. The columns were washed with different CVs of IMAC wash buffer containing 1 w % of detergent 1 or hybrid detergent 3, such as 0, 1, 5, 10, 20, or 40. Subsequently, the columns were washed with 1 mL of an IMAC wash/elute buffer mixture (9/1 v/v, 2×critical aggregation concentration of detergent 1 (nb. detergent 1 used irrespective of the detergent used in the wash buffer), pH=8). The proteins were eluted with 650 μL IMAC elute buffer (50 mM Tris, 200 mM NaCl, 20 mM imidazole, 10% v/v glycerol, 2×critical aggregation concentration of detergent 1 (nb. detergent 1 used irrespective of the detergent used in the wash buffer), pH=8). The relative LPS concentration was monitored by SDS PAGE silver stain analysis and the samples were concentrated to a final volume of 20 to 30 μL using Amicon® Ultra 0.5 mL centrifugal filters. The sample buffer was exchanged to ammonium acetate solution (200 mM, PH=6.8, 2×critical aggregation concentration of detergent 1 (nb. detergent 1 used irrespective of the detergent used in the wash buffer)) using 75 μL Zeba™ Spin Desalting columns (MWCO=7 kDa, Thermo Fisher Scientific). The buffer exchange was done twice to give a delipidated sample

### Mass Spectrometry Analysis of Delipidated Proteins

The samples were analysed using a Q-Exactive mass spectrometer using the following instrumental parameters:
Injection flatapole: 7.9 V
Inter flatapole lens: 6.9 V
Bent flatapole: 5.9 V
Transfer multipole: 4 V
Capillary voltage: 1.2 kV
Source temperature: 100° C.
Voltage applied to the C-trap entrance lens: 5.8 V
In-source trapping voltage: 0-200 V
Higher-energy collisional dissociation (HCD) cell voltage: 100-200 V
HCD cell pressure: 9×10⁻¹⁰ mBar
Noise level parameter: 3
Microscans: 10
Resolution: 17,500

Intensities of the apo states and protein-lipid complexes were extracted from Xcalibur V2.2. Molecular masses of membrane protein ions and bound ligands were calculated using Qual Browser (Thermo Xcalibur 4.1.31.9) and Origin V9.1. Relative intensities of apo states and protein-lipid complexes as well as Zₐᵥₑ values of membrane protein ions were calculated by means of Navia Beta v0.5 (https://d-que.github.io/navia/) and Origin V9.1.

### Top-Down Analysis of Membrane Proteins

The Q-Exactive mass spectrometer was operated with negative nESI polarity for top-down mass spectrometry using the following parameters:
Injection flatapole: −5 V
Inter flatapole lens: − 4 V
Bent flatapole: − 2 V
Transfer multipole: 0 V
Capillary voltage: 0.9 kV
Source temperature: 200° C.
Source fragmentation 0 V
In-source trapping: 200 V
HCD collision energy: 0 V
HCD cell nitrogen pressure: 8×10⁻¹⁰ mbar
Noise level parameter: 3
Microscans: 5
Resolution: 17,500 at m/z=200 (transient time of 64 ms)

The S-lens RF was set to 100%, and an m/z range set to 350 to 20,000. To dissociate ligands bound to AqpZ, MS/MS spectra were obtained upon isolating the ion population of the AqpZ charge state 17—using the quadrupole (m/z range=5,800 to 6,000) selection between 5,800 and 6,000 m/z in order to and increasing the HCD voltage to 200 V for detergent 3 or 50 V for detergent 5. To facilitate the detection of dissociated lipids, nitrogen pressure in the HCD cell was decreased to 3×10⁻¹⁰ mbar and the resolution was increased to 200,000 at m/z=200. The noise level was set to 3 rather than the default value of 4.64 and the number of microscans was increased to 10 rather than 5.

### Determination of the Critical Aggregation Concentration of Hybrid Detergents

The critical aggregation concentration (CAC) of the hybrid detergents was determined following a protocol previously published in Urner, L. H. et al. 2020, Nat. Commun. 11, 564; and in 2 Urner, L. H. et al. 2020, Chem. Sci. 11, 3538-3546. Specifically, the CAC of the hybrid detergents was determined by dynamic light scattering (DLS). Serial dilutions of detergents were prepared in MilliQ water with concentrations ranging from 10⁻⁸ and 10⁻² mol· L⁻¹. The samples were filtered (0.22 μm, regenerated cellulose) and equilibrated for one day at room temperature (approximately 22° C.). The samples were analysed in cuvettes (Quartz Suprasil, width×length: 2 mm×10 mm) using a Zetasizer Nano-ZS ZEN3600 (Malvern, UK). The instrumental parameters were as follows:
Material: Polystyrene Latex
Dispersant: Water
Sample viscosity parameters: Use dispersant viscosity as sample viscosity
Temperature: 22.5° C.
Equilibration time: 120 seconds
Cell type: Quarz cuvettes
Measurement angle: 173° Backscatter
Measurement duration: Manual
Number of runs: 11
Run duration: 10 seconds
Number of measurements: 3
Delay between the measurements: 0 seconds
Data processing: General purpose (normal resolution)

The derived count rate values obtained from three measurements per concentration were averaged. The unit of the derived count rate is kilo counts per second (kcps). The logarithm of the derived count rate was plotted against the logarithm of the concentration. The double logarithmic plots showed two characteristic regions: (1) a flat region with low count rates at lower concentrations of hybrid detergent and (2) a linear growth of the count rate at higher concentrations of hybrid detergent. Both regions were fitted to linear functions and the intersection was taken as the CAC value (see Skhiri, Y. et al. 2012, Soft Matter, 8, 10618-10627).

### Circular Dichroism

Circular dichroism (CD) experiments were conducted following the procedure published in Urner et al., Nature Communications, 2020, 11, 562 according to which protein solutions obtained upon IMAC with DDM are transferred into detergent-containing CD spectroscopy buffer (100 mM NH₄(HCO₃), pH=8) using desalting columns (column volume=5 mL, GE Healthcare, product number: GE29-0486-84). Specifically, the columns were washed with water (15 mL) and equilibrated with CD spectroscopy buffer (10 mL) containing detergent of interest (i.e. OG, C8E4, 1, 2, 3, 4, 5 or 6 at 2×critical aggregation concentration. Protein solutions obtained upon IMAC (˜0.5 mL) were injected manually into the columns using syringes. The proteins were eluted with CD spectroscopy buffer (10 ml of 100 mM ammonium bicarbonate) and fractions were collected (fraction size=1 mL). Protein-containing fractions were identified by UV spectroscopy, combined, and concentrated to a final protein concentration of 2-8 μM. So-obtained protein solutions were loaded into cuvettes (Quartz Suprasil, volume=300 μL, layer thickness=1 mm). The CD spectrometer (Chirascan, USA) was purged with nitrogen overnight and turned on 30 min before use together with the sample cooler. The following experimental parameters were used:
Temperature: 22.5° C.
Wavelength range: 200-260 nm
Step size: 0.5-1 nm
Scan speed: 0.5 s/point
Bandwidth: 1 nm
Repeats per sample: 4

The average CD intensity of four scans was plotted against the wavelength. Detergent-containing CD spectroscopy buffers were used as blanks. Data were acquired with Pro-Data Chirascan V4.5 and analyzed with Origin V9.1.

### Example 1: Preparation of Hybrid Detergents

The following building blocks were used to prepare hybrid detergents:

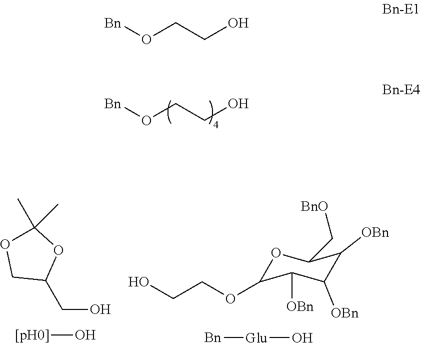

Protected forms of the head groups for detergents were synthesised via the following reaction:

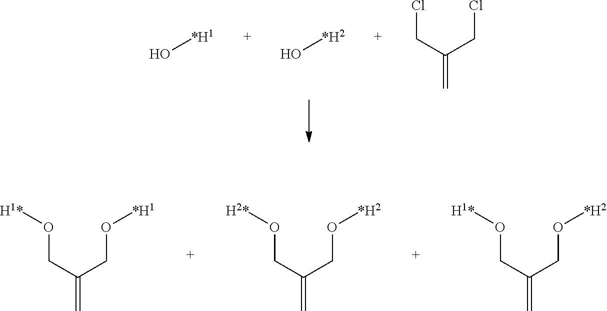

where * represents that the head groups H¹ and H² are in a protected form.

The following detergent library was prepared from the protected head groups:

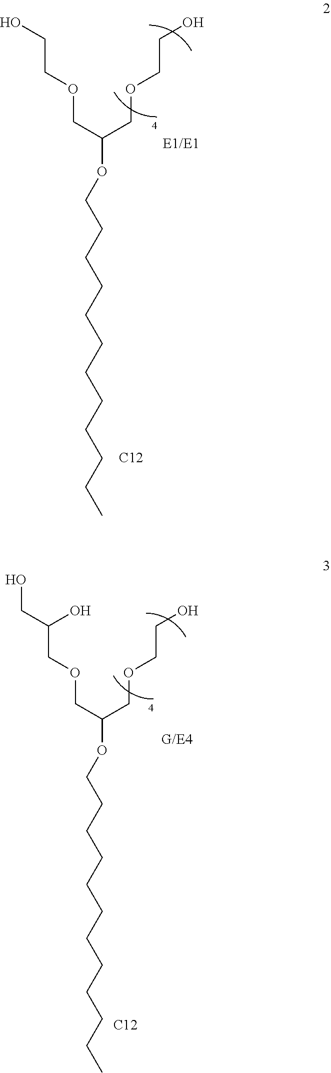

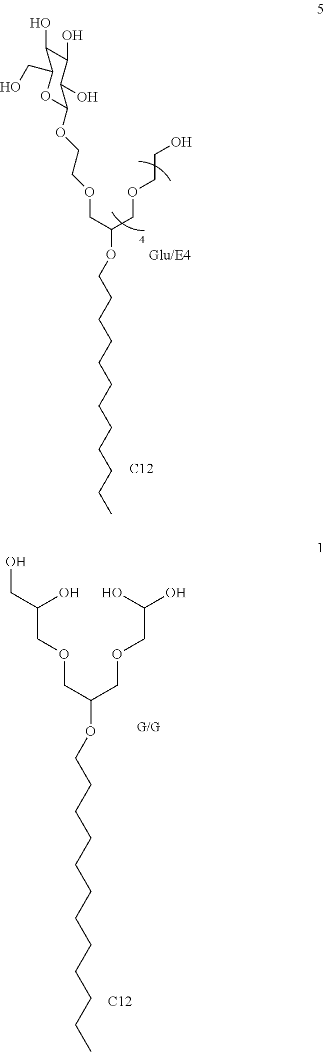

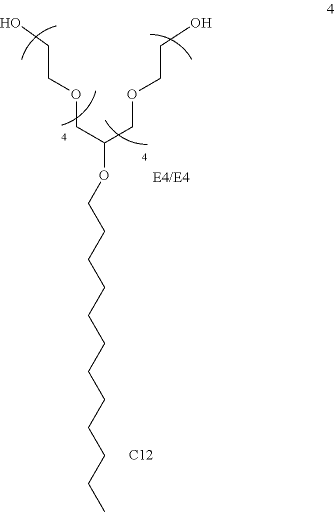

Within the detergent library, detergents 2, 3 and 5 are hybrid detergents.

In a first preparation method, the protected head groups were separated by column chromatography and converted into the detergents. In a second preparation method, the protected head groups were converted into detergents mixture and then separated by column chromatography to give the detergents shown above.

### Example 2: Properties of the Hybrid Detergents

The hydrophobic-lipophilic balance (HLB) of detergents 1-5 was calculated according to the following equation:

$$HLB = 20 \cdot ( 1 - \frac{{MW}_{tail}}{MW} ) \text{?}$$

$$\text{?} \text{indicates text missing or illegible when filed}$$

where: MW represents the molecular weight of the detergent; and
MWₜₐᵢₗ represents the molecular weight of the hydrophobic tail.

The packing parameter, p, of the detergents was calculated according to the following equation:

$$p = \frac{\text{?}}{\text{?}} \text{?}$$

$$\text{?} \text{indicates text missing or illegible when filed}$$

where: Vₜₐᵢₗ represents the volume of the hydrophobic tail;
Iₜₐᵢₗ represents the length of the hydrophobic tail; and
A_{head} represents the area of the head group that is occupied at the interface between the detergent aggregate and solvent.

For comparison, the following commercially available saccharide detergent and polyethelene glycol detergent were also analysed (though commercially available, these detergents can also be synthesised using known methods e.g. Naskar et al., Journal of Physical Chemistry C, 2015, 119, 20985-20992; Mladenoska, Food Technol. Biotechnol., 2016, 54, 211-216):

The HLB and p values are shown in the following table:

| | MW | MWtail | | A_{head} | Vₜₐᵢₗ | Iₜₐᵢₗ | |
|---|---|---|---|---|---|---|---|
| detergent | (g/mol) | (g/mol) | HLB | (nm²) | (nm³) | (nm) | p |
| OG | 229.4 | 113.2 | 10.1 | 0.715 | 0.147 | 1.0 | 0.20 |
| C8E4 | 306.5 | 113.2 | 12.6 | 0.863 | 0.147 | 1.0 | 0.17 |
| 1 | 408.5 | 169.3 | 11.7 | 1.023 | 0.215 | 1.6 | 0.13 |
| 2 | 480.6 | 169.3 | 12.9 | 1.118 | 0.215 | 1.6 | 0.12 |
| 3 | 510.7 | 169.3 | 13.3 | 1.266 | 0.215 | 1.6 | 0.10 |
| 4 | 612.8 | 169.3 | 14.4 | 1.536 | 0.215 | 1.6 | 0.09 |
| 5 | 642.8 | 169.3 | 14.7 | 1.442 | 0.215 | 1.6 | 0.09 |

### Example 3: Phospholipid Delipidation at Low Detergent Concentration

Translocator protein (TSPO), ammonium channel (AmtB) and aquaporine channel (AqpZ) membrane proteins were provided in the form of n-dodecyl-β-D-maltopyranoside (DDM) detergent aggregates. Detergent exchange was carried out using the detergents of Example 1 to give solutions having 2×critical aggregation concentration (CAC) of the detergent. The solutions were analysed using mass spectrometry.

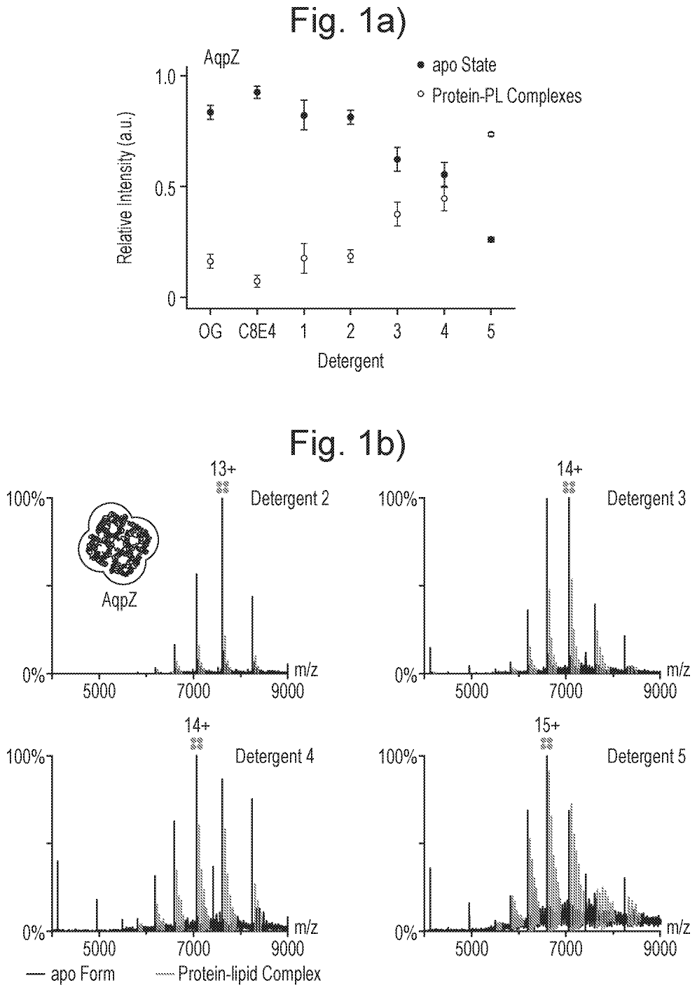

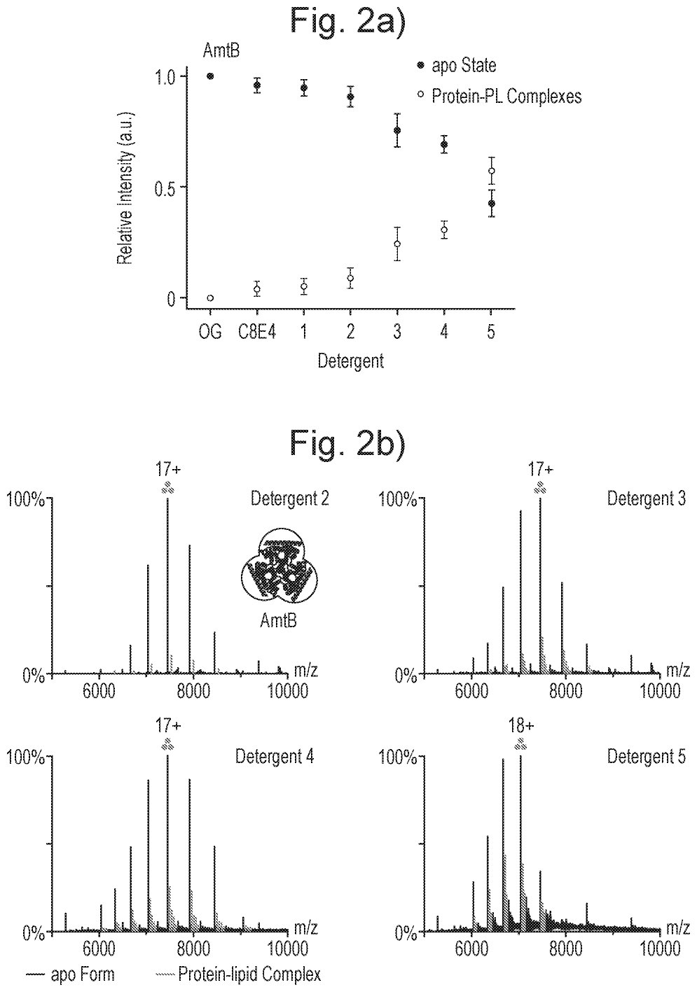

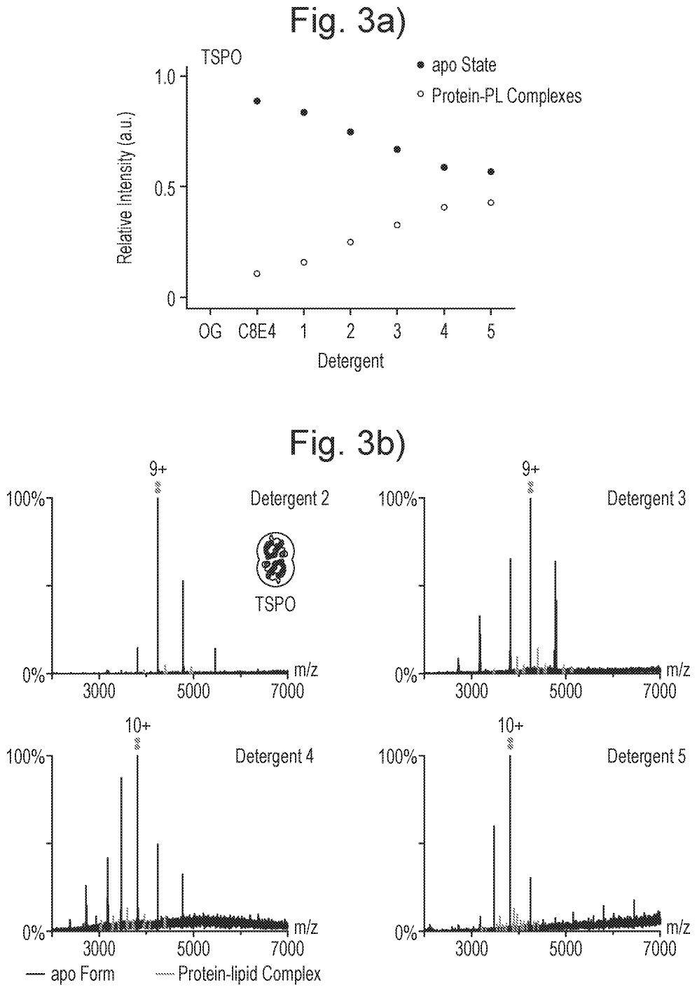

A plot of the relative intensities of the apo states and protein-phospholipid complexes against detergent and the mass spectrometry spectra are shown in FIG. 1 for AqpZ, FIG. 2 for AmtB and FIG. 3 for TSPO. Detergents 1-5 are plotted in the order of increasing HLB.

It can be seen that phospholipids were gradually removed from each of the membrane proteins using the detergent library described in Example 1, with less delipidation occurring as the HLB of the detergent increases and the p value decreases. Notably, even though the strongly delipidating head groups of the OG and C8E4 detergents are combined in detergent 5, detergent 5 is less delipidating towards phospholipids than either OG or C8E4. Similarly, modifying detergent 1 by replacing one of the dendritic head groups with a head group from strongly delipidating C8E4 to gives a detergent 3 which is less delipidating towards phospholipids than detergent 1. This demonstrates that a detergent which has a hybrid head group can be used to provide a surprisingly gentle delipidating environment for a membrane protein.

No membrane protein complexes with lipopolysaccharides, or unbound lipopolysaccharides, were detected in the mass spectrometry experiments on TSPO, AmtB and AqpZ. Further experiments were conducted in which delipidated AmtB was incubated with lipopolysaccharides. AmtB-lipopolysaccharide complexes were subsequently identified using mass spectrometry. This implies that the lipidome that is co-purified with a membrane protein is biased by the lipidome that surrounds the protein in its native membrane. Since phospholipids and not lipopolysaccharides are identified as a binding partners upon purification of AmtB, TSPO and AqpZ, it is likely that these membrane proteins sit in a phospholipid-rich lipid environment in their native membranes.

Further investigations were carried out to determine whether the detergent family prepared in Example 1 could be used to gradually delipidate lipopolysaccharides from membrane proteins. Specifically, large-conductance mechanosensitive channel (MsCl) membrane protein was provided in the form of n-dodecyl-β-D-maltopyranoside (DDM) detergent aggregates. Detergent exchange was carried out using the detergents of Example 1 to give solutions having 2×critical aggregation concentration (CAC) of the detergent. The solutions were analysed using mass spectrometry.

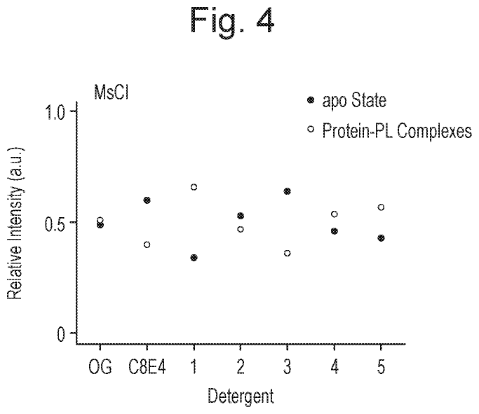

A plot of the relative intensities of the apo states and protein-lipopolysaccharides complexes against detergent and the mass spectrometry spectra are shown in FIG. 4. As before, detergents 1-5 are plotted in the order of increasing HLB.

It can be seen that lipopolysaccharides were partially removed from the membrane protein using the detergent library described in Example 1. Unlike with phospholipids, the degree of delipidation of lipopolysaccharides was largely independent from the HLB and p value of the detergent. As a consequence, proteins that preferentially bind to phospholipids can be distinguished from those that preferentially bind to lipopolysaccharides.

### Example 4: Lipopolysaccharide Delipidation at High Detergent Concentration

To achieve lipopolysaccharide delipidation, samples of multidrug efflux pump subunit (AcrB), and ATP-binding cassette transporter (BtuCD) were provided in the form of DDM detergent aggregates. The samples were loaded onto immobilized metal affinity chromatography (IMAC) columns. Different column volumes of an IMAC wash buffer containing 1% by weight (approximately 50×CAC) of detergent 1 or hybrid detergent 3 were used to wash the columns, to determine how many column volumes of the IMAC wash buffer are needed to delipidate lipopolysaccharides from the proteins.

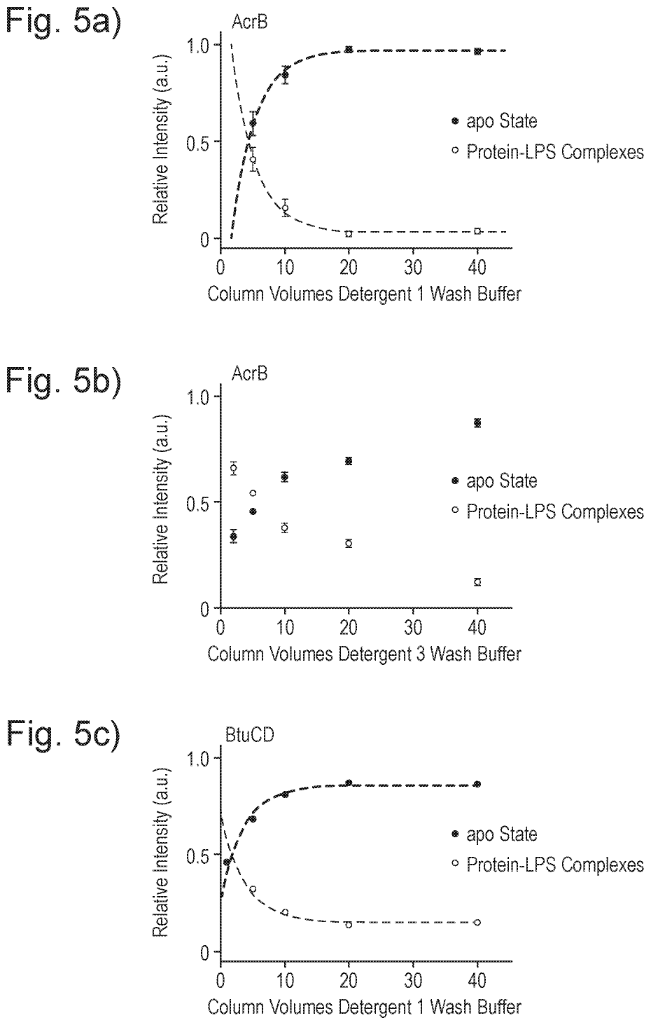

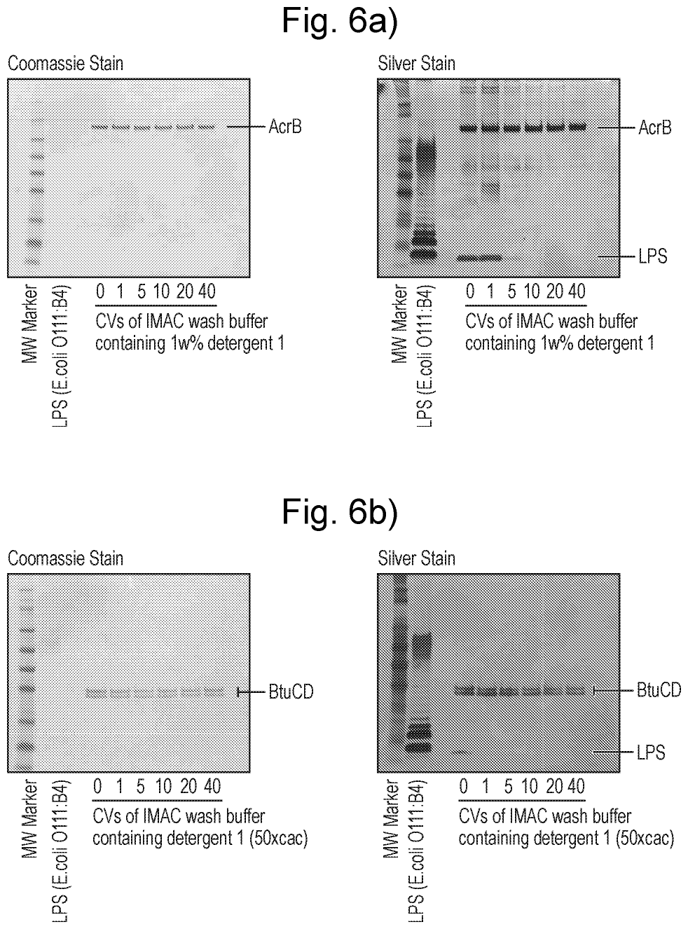

The proteins were eluted from the column using an IMAC elute buffer containing 2×CAC of detergent 1 (detergent 1 was used irrespective of the IMAC wash buffer detergent). The eluted samples were analysed using mass spectrometry to determine the relative amount of apo and bound protein, and SDS PAGE silver stain analysis (Coomassie stain being more sensitive to proteins, and silver stain to proteins and lipopolysaccharides) to measure the relative lipopolysaccharide concentration. The results are shown in FIGS. 5 and 6 respectively.

It can be seen from FIG. 5 that approximately 30 column volumes of the detergent 1 IMAC wash buffer were needed to achieve maximum delipidation of the membrane protein-lipopolysaccharide complexes, and that more column volumes of hybrid detergent 3 were required. For both detergents, the relative amounts of protein-lipopolysaccharide complexes and protein in the apo state may be finetuned by using different volumes of a concentrated detergent solution.

Further experiments were conducted in which delipidated AcrB was incubated with phospholipids. AcrB-phospholipid complexes were subsequently identified using mass spectrometry. This provides further evidence that the lipidome that is co-purified with a membrane protein is dependent on the lipidome that surrounds the protein in its native membrane, and it is likely that AcrB (and BtuCD) sit in a lipopolysaccharide-rich lipid environment in their native membranes.

### Example 5: Impact of Critical Aggregation Concentration and Average Charge State on Delipidation

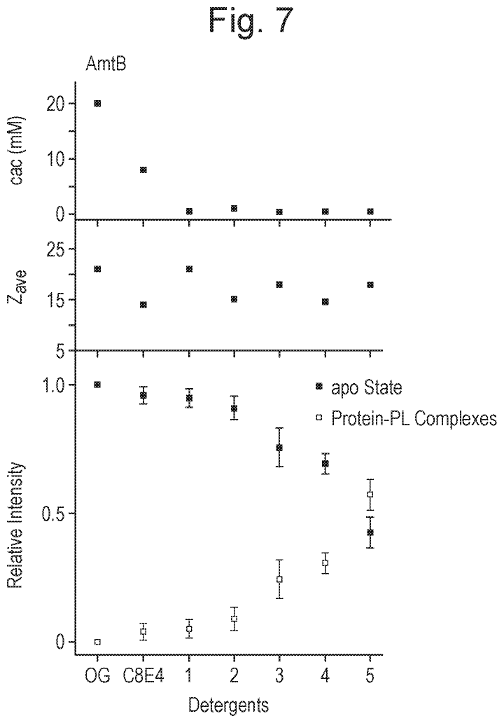

To investigate whether other experimental conditions were responsible for the results obtained above, the degree to which the membrane protein AmtB was delipidated was assessed against the CAC of the detergent family of Example 1 and average charge state (Zₐᵥₑ) of the detected proteins. A plot is shown in FIG. 7.

It can be seen that the relative intensities of apo state and protein-PL complexes are independent from both the detergent concentration used during delipidation and Zₐᵥₑ values of membrane protein ions. This supports the hypothesis that differences in membrane protein delipidation are due to the different detergent proteomicelles formed in solution.

### Example 6: Interrogation of a Membrane Protein Lipidome

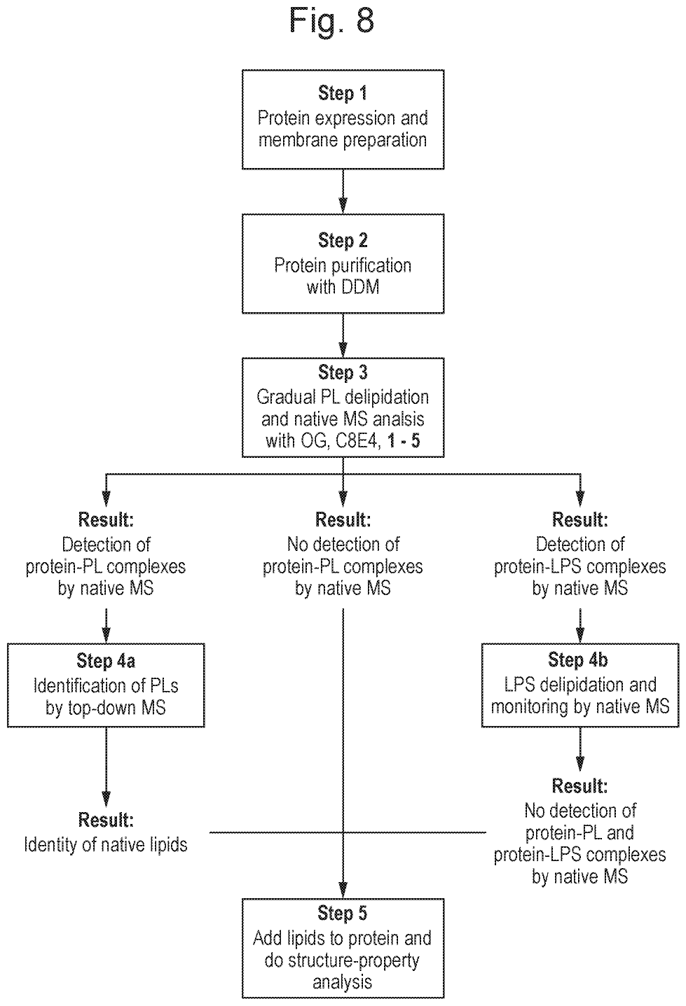

Example 3 demonstrates that it is possible to distinguish between phospholipid and lipopolysaccharide ligands using the detergent family of Example 1; while less phospholipid delipidation occurring as the HLB of the detergent increases and the p value decreases, lipopolysaccharide delipidation is independent of these properties. Example 4 demonstrates that gradual lipopolysaccharide delipidation can, however, be achieved by using increasing volumes of a relatively strong detergent solution. These discoveries have enabled an idealised workflow to be devised for interrogating the lipidome of a membrane protein of interest. The workflow is depicted in FIG. 8.

By following the workflow, a structure-property analysis of membrane-protein-lipid interactions is facilitated.

### Example 7: Preparation and Use of a Further Hybrid Detergent

The following hybrid detergent was prepared:

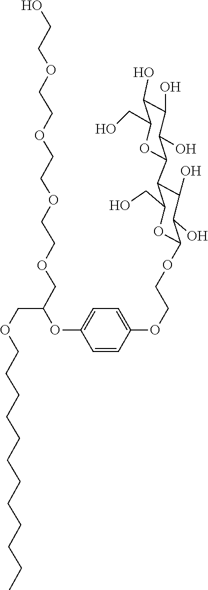 [SMILES: CCCCCCCCCCCCOCC(COCCOCCOCCOCCO)Oc1ccc(OCCOC2OC(CO)C(C3OC(CO)C(O)C(O)C3O)C(O)C2O)cc1]

The hybrid detergent includes a head group derived from DDM and a head group derived from C8E4.

Different solutions of the membrane protein AqpZ were prepared by extracting the protein from its native membrane using a 1% w/v detergent solution and then purifying the samples using IMAC. The following solutions were prepared: one containing the hybrid detergent, one containing DDM, one containing C8E4 and one containing a mixture of C8E4 and DDM. AgpZ precipitated from the solutions containing C8E4, even when DDM was also present. This suggests that the denaturing properties of polyethylene glycol detergents remain even in the presence of a non-denaturing detergent. Surprisingly, AgpZ was stable in the solution containing the hybrid detergent even though a polyethylene glycol is present as a head group.

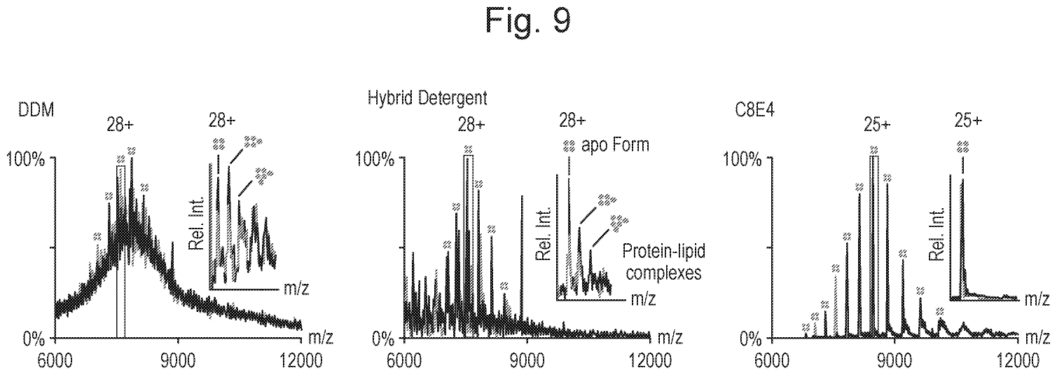

The suitability of the hybrid detergent in nano-electrospray ionization was investigated using a Q Exactive mass spectrometer. The results are shown in FIG. 9, along with results obtained using DDM and C8E4.

Each of the detergents revealed charge states corresponding to the apo form of AqpZ though, as expected, relatively low levels of delipidation were observed with DDM. Lipid-bound states were only detected in the cases of DDM and the hybrid detergent, with C8E4 fully delipidating the membrane protein.

### Example 8: Top-Down Down Identification of Lipids

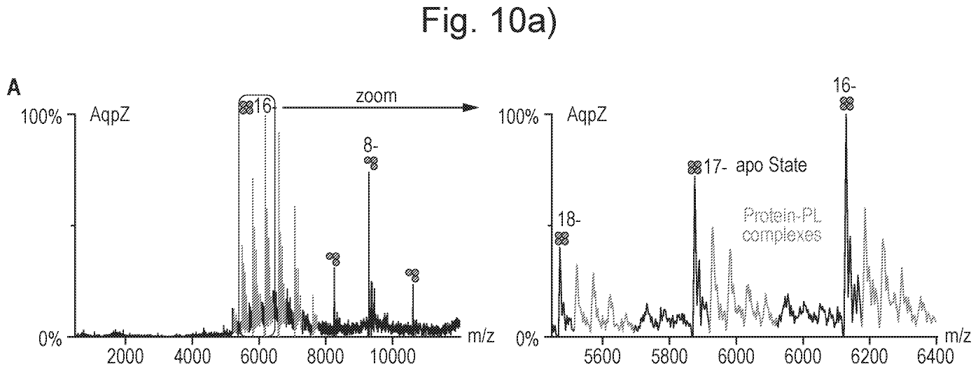

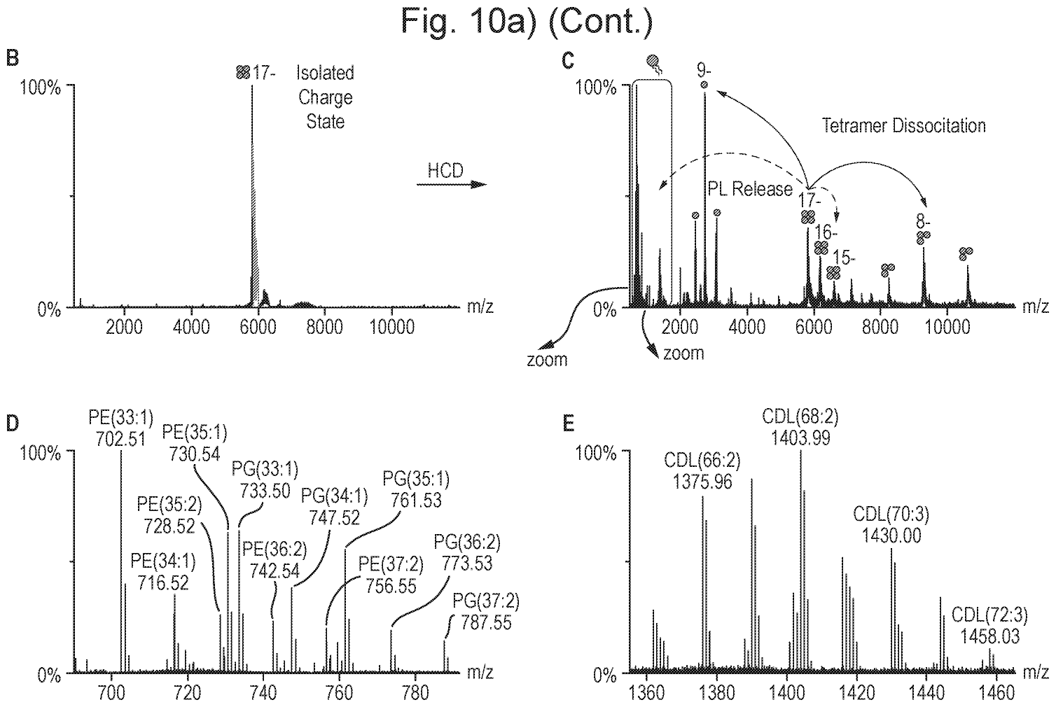

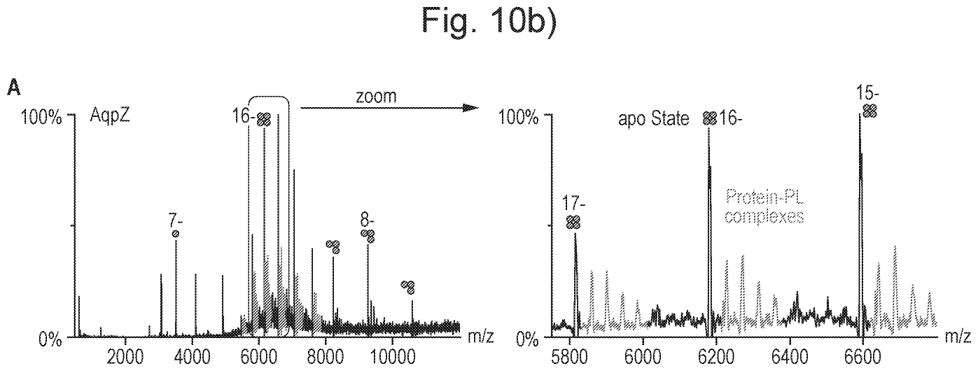

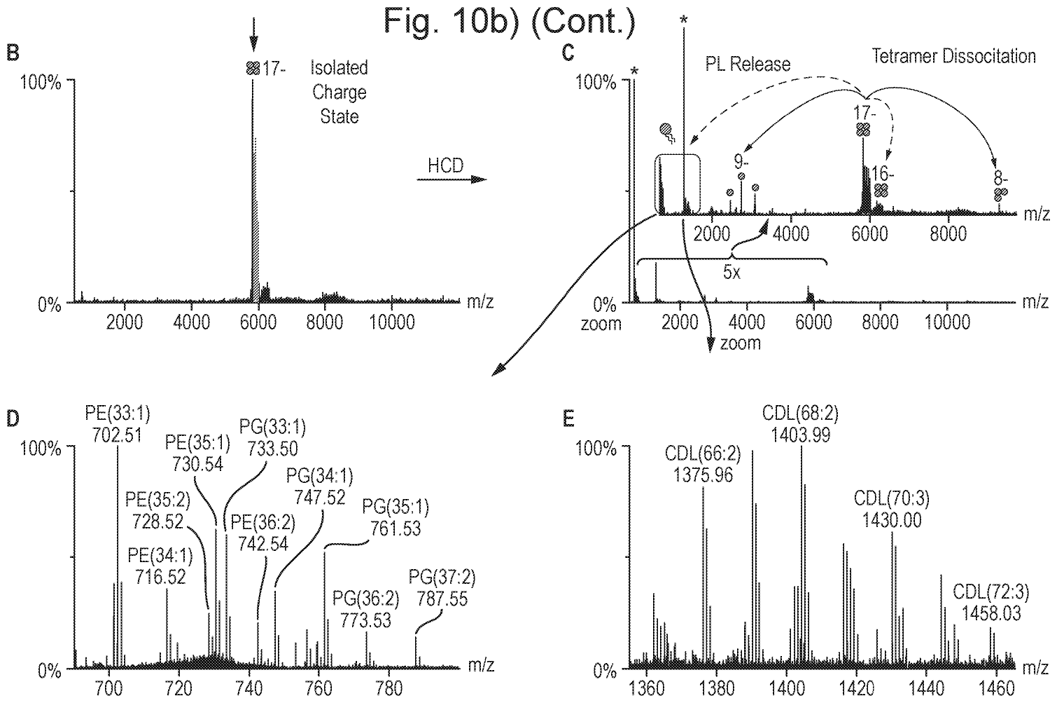

The nature of the phospholipids bound to the membrane protein AqpZ was further interrogated using top-down mass spectrometry. As in Example 3, the protein was first purified with DDM then delipidated using either hybrid detergent 3 or hybrid detergent 5. The results are shown in FIG. 10a for detergent 3 and FIG. 10b for detergent 5.

Each of the figures shows nESI mass spectra for the protein AqpZ obtained during different stages of ligand identification using native top-down MS. First, the detergent micelle is removed to obtain a mass spectrum of the protein, including the apo form and protein-ligand complexes (A). Second, a charge state is selected, including signals of the apo state and protein-ligand complexes (B). Third, the charge state is activated by higher-energy collisional activation (HCD) to dissociate protein-ligand complexes and detect dissociated ligands and protein subunits (C). Analysing the mass of ions with lower mass-to-charge ratios (m/z), e.g. 690-800 and 1350-1470 m/z, confirms that the following phospholipids dissociated from AqpZ: phosphatidylglycerol (PG), phosphatidylethanolamine (PE), and cardiolipin (CDL) (D-E).

### Example 9: Preservation of Secondary Membrane Protein Structure

Circular dichroism experiments were carried out to determine the extent to which the secondary structure of membrane proteins is preserved in solutions containing 2×CAC of the detergent family of Example 1, OG, C8E4 and and the further following detergent:

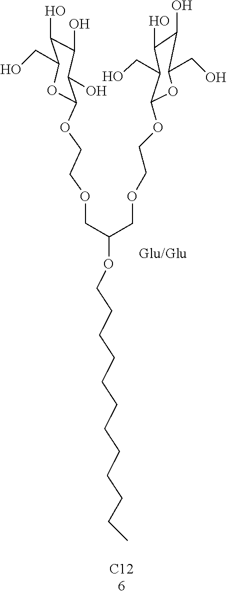 [SMILES: CCCCCCCCCCCCOC(COCCOC1OC(CO)C(O)C(O)C1O)COCCOC1OC(CO)C(O)C(O)C1CO]

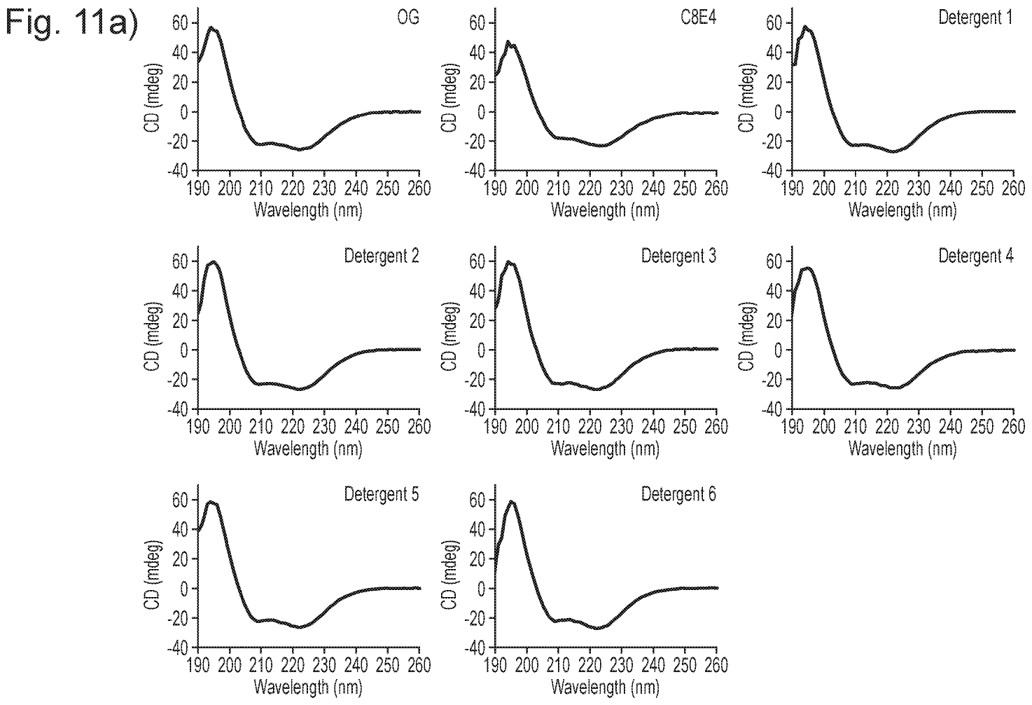

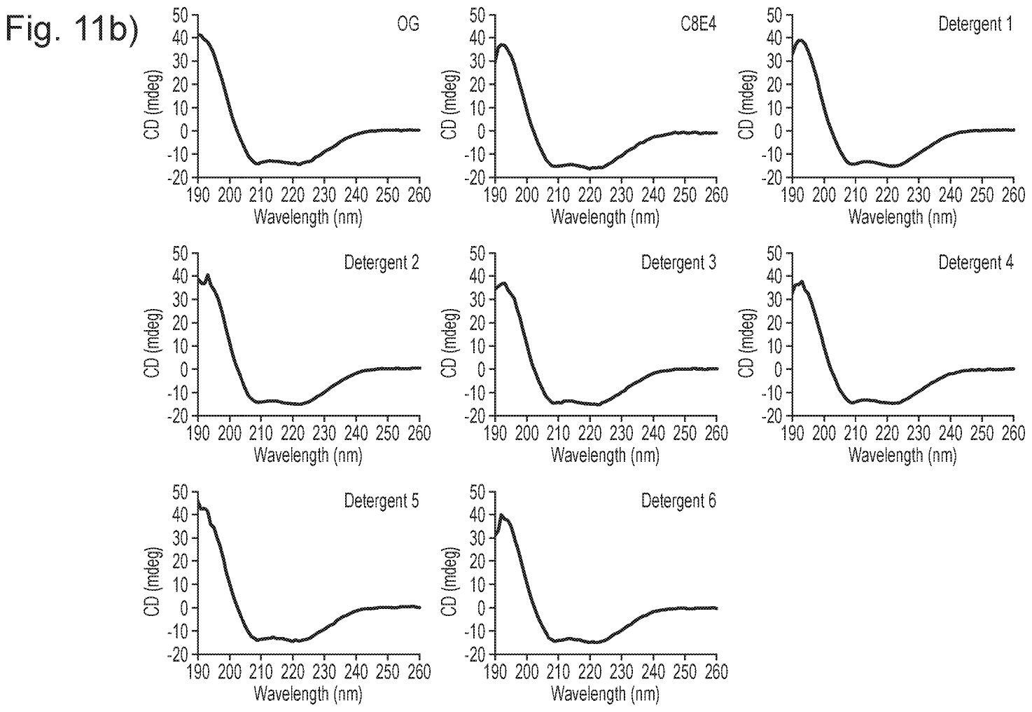

The membrane proteins TSPO and AmtB were used in the experiments. The results are shown in FIG. 11a for TSPO and FIG. 11b for AmtB.

Minima in intensity at 208 nm and 222 nm are expected if the alpha-helical structures in TSPO and AmtB are preserved. These are observable in each of the spectra thereby demonstrating that the detergent family 1 of Example 1, including the hybrid detergents, may preserve the secondary structure of membrane proteins.

1. A method of detecting a protein by mass spectrometry, wherein the method comprises:
(a) providing a solution comprising a hybrid detergent and a protein;
(b) providing a mass spectrometer comprising a nanoelectrospray ionisation source, a mass analyser and a detector;
(c) vaporising the solution using the nanoelectrospray ionisation source;
(d) ionising the protein;
(e) resolving the ionised protein using the mass analyser; and
(f) detecting the resolved protein using the detector;
wherein the hybrid detergent comprises a hybrid head group linked to a hydrophobic tail, wherein the hybrid head group comprises a first hydrophilic group and a second hydrophilic group which is different from the first hydrophilic head group, wherein each of the first and second hydrophilic groups is derived from a polyol or contains a charged group.

2. The method of claim 1, wherein the polyol is selected from diols (e.g. ethylene glycol), triols (e.g. glycerol), saccharides (e.g. hexoses, pentoses or tetroses) in cyclic or linear form, or oligomers thereof.

3. The method of claim 2, wherein the first and second hydrophilic groups are selected from: 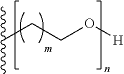
where m is 1-3, and n is 1-8; 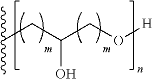
where each m is independently 1-3, and n is 1-5; 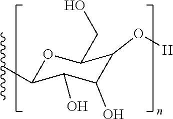
where n is 1-5; and 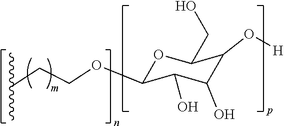
where m is 1-3, n is 1-5 and p is 1-5.

4. The method of claim 1 or claim 2, wherein the charged group is a phosphate-containing group.

5. The method of any preceding claim, wherein the hybrid detergent has the formula: 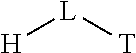
where: H represents the hybrid head group;
L represents a linking group; and
T represents the hydrophobic tail.

6. The method of any preceding claim, wherein the hybrid head group has the structure: 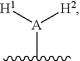
where: A represents a trivalent group;
H¹ represents the first hydrophilic group; and
H² represents the second hydrophilic group,
and preferably wherein the hybrid head group has the structure:

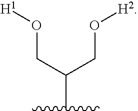

7. The method of any preceding claim, wherein the hybrid head group is joined to the hydrophobic tail by a linking group, L, selected from: hydrocarbylene, heterocyclylene, O, S, NR′, NR′—O, C(O)NR′, OC(O)NR′, OC(O)O, NR′C(O)NR′, NR′C(S)NR′, C(NR′)NR′, C(O), C(O)O, S(O)₂, S(O), S(O)₂O, S—S, CR′═N, CR′═N—NR′, C═N—NR′C(O), and combinations of up to three of these groups, where each R′ is independently selected from H, C₁₋₄ alkyl and C₁₋₄ alkoxy; and preferably wherein the linking group is selected from: O, S, C(O)O, O—C₁₋₄ alkylene-aryl and OC(═O)NR′.

8. The method of any preceding claim, wherein the hydrophobic tail comprises a C₆₋₁₀₀ alkyl group in which one or more methylene groups may be independently replaced by a unit selected from: C₂₋₆ alkenylene, C₂₋₆ alkynylene, arylene, O, S, NR″, NR″—O, C(O)NR″, OC(O)NR″, OC(O)O, NR″C(O)NR″, NR″C(S)NR″, C(NR″)NR″, C(O), S(O)₂, S(O), S(O)₂O, S—S, CR″═N, CR″═N—NR″, C═N—NR″C(O), where each R″ is independently selected from H, C₁₋₄ alkyl and C₁₋₄ alkoxy.

9. The method of any preceding claim, wherein the hybrid detergent has:
a total number of carbons of up to 60, preferably up to 50, and more preferably up to 40; and/or
a molecular weight of up to 1,000 Da, preferably up to 800 Da, and more preferably up to 650 Da.

10. The method of any preceding claim, wherein the protein is detected in the form of a complex with a lipid, such as a phospholipid or a lipopolysaccharide.

11. The method of any preceding claim, wherein the hybrid detergent is present in the solution at a concentration which is greater than or equal to the critical aggregation concentration (CAC) of the hybrid detergent in said solution, and preferably wherein:
the hybrid detergent is present in the solution at a concentration which is from 1.5 to 3 times the CAC, and more preferably from 1.75 to 2.5 times the CAC; or
the hybrid detergent is present in the solution at a concentration which is from 35 to 65 times the CAC, and more preferably from 45 to 55 times the CAC.

12. The method of any preceding claim, wherein the protein is detected substantially intact.

13. The method of any preceding claim, wherein the structure or conformation of the protein is characterised.

14. The method of any preceding claim, wherein the protein is a membrane protein.

15. A method of preparing a protein sample, wherein the method comprises:
(i) providing a solution which comprises an extraction detergent aggregate in which a protein is contained; and
(ii) contacting the extraction detergent aggregate with a hybrid detergent to give a solution which comprises a hybrid detergent aggregate in which the protein is contained, wherein the hybrid detergent has a structure as defined in claim 9.

16. The method of claim 15, wherein the method comprises extracting the protein from its native membrane by contacting the protein with an extraction detergent to form the extraction detergent aggregate.

17. The method of claim 15 or claim 16, wherein the protein is present in the extraction detergent aggregate in a lipidated form.

18. The method of any of claims 15 to 17, wherein detergent exchange step (ii) is carried out using size exclusion chromatography (SEC).

19. The method of any of claims 15 to 18, wherein the extraction detergent is n-dodecyl β-D-maltoside.

20. A hybrid detergent as defined in claim 9.

21. A solution comprising a hybrid detergent as defined in claim 20 and a protein, wherein the hybrid detergent preferably forms a detergent aggregate in which the protein is contained.

22. A protein delipidation kit, said kit comprising at three different detergents, wherein at least one of the detergents is a hybrid detergent as defined in any of claims 1 to 9, and wherein the kit preferably comprises instructions for carrying out a method according to any of claims 1 to 19.

23. A method of interrogating the lipidome of a protein of interest, said method comprising:
providing at least three solutions comprising a detergent and the protein, a different detergent being used in each solution;
providing a mass spectrometer comprising a nanoelectrospray ionisation source, a mass analyser and a detector, and for each of the solutions:
vaporising the solution using the nanoelectrospray ionisation source;
ionising the protein;
resolving the ionised protein using the mass analyser;
detecting the resolved protein using the detector; and
determining the degree of lipidation in the detected protein;
calculating at least one of the hydrophobic-hydrophilic balance (HLB) and the packing parameter (p value) of each of the detergents; and
correlating the HLB and/or p value of the detergents with the degree of lipidation in the detected protein.

24. The method of claim 23, wherein at least two of the solutions comprise a hybrid detergent as defined in any of claims 1 to 9.

25. The method of claim 23 or claim 24, wherein the method comprises detecting the protein in a lipidated state in at least one, preferably at least two, and more preferably at least three, of the at least three solutions.
